(19) United States
(12) Patent Application Publication
(10) Pub. No.: US 2015/0209362 A1
(43) Pub. Date: Jul. 30, 2015

(54) PHARMACEUTICAL COMBINATIONS COMPRISING A PYRIDO [4,3-D] PYRIMIDINE DERIVED HSP90-INHIBITOR AND A HER2 INHIBITOR

(71) Applicants: Carlos Garcia-Echeverria, Basel (CH); Michael Rugaard Jensen, Basel (CH)

(72) Inventors: Carlos Garcia-Echeverria, Basel (CH); Michael Rugaard Jensen, Basel (CH)

(73) Assignee: Novartis AG, Basel (CH)

(21) Appl. No.: 14/621,808

(22) Filed: Feb. 13, 2015

Related U.S. Application Data
(63) Continuation of application No. 14/284,240, filed on May 21, 2014, now abandoned.
Application No. 14/284,240 is a continuation of application No. 13/466,190, filed on May 8, 2012, now abandoned.
Application No. 13/466,190 is a continuation of application No. 13/131,298, filed on May 26, 2011, now abandoned.

(30) Foreign Application Priority Data
Nov. 28, 2008 (EP) 08170255.7
Nov. 28, 2008 (EP) 08170261.5

(51) Int. Cl. A61K 31/5377; A61K 39/395

(52) U.S. Cl. CPC A61K 31/5377; A61K 39/39558; A61K 2039/505

(57) ABSTRACT

A pharmaceutical combination comprising an Hsp90 inhibitor and an HER2 inhibitor, and methods of using the combination to treat proliferative disorders.

This application is a continuation of application Ser. No. 13/131,298 filed May 26, 2011 which is a national stage of International Application No. PCT/EP2009/065861 filed on Nov. 25, 2009 which claims priority under 35 USC 119 to European Application Numbers 08170255.7, filed Nov. 28, 2008, and 08170261.5, filed Nov. 28, 2008, the contents of all four applications are incorporated by reference.

### BACKGROUND OF THE INVENTION

1. Field of the Invention

The present invention is directed to a pharmaceutical composition comprising an Hsp90 inhibitor and one or more pharmaceutically active agent, e.g. a HER2 inhibitor, and the uses of such a composition for the treatment of disease, including proliferative diseases.

2. Related Background Art

In spite of numerous treatment options for proliferative disease patients, there remains a need for effective and safe antiproliferative agents and a need for their use in combination therapy.

Heat shock protein 90 (Hsp90) is recognized as an anti-cancer target. Hsp90 is a ubiquitous, highly abundant (1-2% of the total cellular protein), essential protein which functions as a molecular chaperone to ensure the conformational stability, shape and function of client proteins.

Among the stress proteins, Hsp90 is unique because it is not required for the biogenesis of most polypeptides (Nathan et al., 1997). Its cellular targets, also called client proteins, are conformationally labile signal transducers that play a critical role in growth control, cell survival and tissue development (Pratt and Toft, 2003). Inhibition of its intrinsic ATPase activity of Hsp90 disrupts the Hsp90-client protein interaction resulting in their degradation via the ubiquitin proteasome pathway. A subset of Hsp90 client proteins, such as Raf, AKT, phospho-AKT and CDK4 are oncogenic signaling molecules critically involved in cell growth, differentiation and apoptosis, processes which are important in cancer cells. The degradation of one or multiple oncoproteins is believed to produce the anti-tumor effects observed with Hsp90 inhibitors.

The Hsp90 family of chaperones is comprised of four members: Hsp90α and Hsp90β both located in the cytosol, GRP94 in the endoplasmic reticulum, and TRAP1 in the mitochondria (Csermely et al., 1998). Hsp90 is the most abundant cellular chaperone, constituting about 1%-2% of total protein (Jakob and Buchner, 1994).

Hsp90 chaperones, which possess a conserved ATP-binding site at their N-terminal domain (Chene, 2002) belong to a small ATPase sub-family known as the DNA Gyrase, Hsp90, Histidine Kinase and MutL (GHKL) sub-family (Dutta and Inouye, 2000). The chaperoning (folding) activity of Hsp90 depends on its ATPase activity which is weak for the isolated enzyme. However, it has been shown that the ATPase activity of Hsp90 is enhanced upon its association with proteins known as co-chaperones (Kamal et al., 2003). Therefore, in vivo, Hsp90 proteins work as subunits of large, dynamic protein complexes. Hsp90 is essential for eukaryotic cell survival and is overexpressed in many tumors.

### BRIEF SUMMARY OF THE INVENTION

It has now been found that a combination comprising at least one Hsp90 inhibitor compound and at least one HER2 inhibitor, e.g. as defined below, has a beneficial effect on proliferative disorders, including without limitation, e.g. solid tumors, e.g. breast cancer.

A pharmaceutical combination according to the invention comprises components (a) and (b), wherein component (a) is an HSP90 inhibitor according to Formula (I)

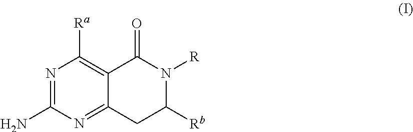

or a stereoisomer, tautomer, pharmaceutically acceptable salt, or prodrug thereof wherein

R^{a} is selected from the group consisting of

(1) hydrogen,

(2) halogen,

(3) hydroxyl,

(4) C₁-C₆ alkoxy,

(5) thiol,

(6) C₁-C₆ alkylthiol,

(7) substituted or unsubstituted C1-C6 alkyl,

(8) amino or substituted amino,

(9) substituted or unsubstituted aryl,

(10) substituted or unsubstituted heteroaryl, and

(11) substituted or unsubstituted heterocyclyl;

R is selected from the group consisting of

(1) hydrogen,

(2) substituted or unsubstituted C1-C6 alkyl,

(3) substituted or unsubstituted C2-C6 alkenyl,

(4) substituted or unsubstituted C2-C6 alkynyl.

(5) substituted or unsubstituted C3-C7 cycloalkyl,

(6) substituted or unsubstituted C5-C7 cycloalkenyl,

(7) substituted or unsubstituted aryl,

(8) substituted or unsubstituted heteroaryl, and

(9) substituted or unsubstituted heterocyclyl;

R^{b} is selected from the group consisting of

(1) substituted or unsubstituted C3-C7 cycloalkyl,

(2), substituted or unsubstituted C5-C7 cycloalkenyl,

(3) substituted or unsubstituted aryl,

(4) substituted or unsubstituted heteroaryl, and

(5) substituted or unsubstituted heterocyclyl; and

with the proviso that when R^{a} is amino, then R^{b} is not phenyl, 4-alkyl-phenyl, 4-alkoxy-phenyl, or 4-halo-phenyl,

or an Hsp90 inhibitor of formula (D),

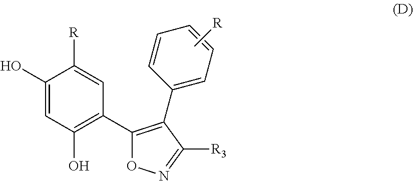

wherein each R independently represents an optional substituent and R₃ represents a carboxamide group and component (b) is an HER2 inhibitor.

The compound according to Formula (I) may be combined with the HER2 inhibitor in a pharmaceutically acceptable carrier. In a method of treating proliferative diseases, an effective amount of the compound according to Formula (I) may be administered to a patient in need thereof in combination with an HER2 inhibitor inhibitor, together or separately, at the same time, or sequentially.

### BRIEF DESCRIPTION OF THE DRAWINGS

FIG. 1 shows the effect of compound I (5-(2,4-Dihydroxy-5-isopropyl-phenyl)-4-4-morpholin-4-ylmethyl-phenyl)-isoxazole-3-carboxylic acid ethylamide also known as AUY922) with trastuzumab in the BT-474 breast cancer xenograft model.

### DETAILED DESCRIPTION OF THE INVENTION

In preferred embodiments of the invention, compounds of formula (III) are provided as the first pharmaceutical component, an Hsp90 inhibitor, in combination with an HER2 inhibitor as the second pharmaceutical component:

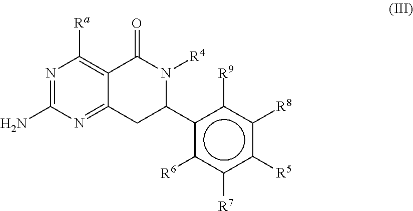

or a stereoisomer, tautomer, pharmaceutically acceptable salt, or prodrug thereof, wherein

wherein R^{a} is selected from the group consisting of

(1) hydrogen,

(2) halogen,

(3) hydroxyl,

(4) C₁-C₆ alkoxy,

(5) thiol,

(6) C₁-C₆ alkylthiol,

(7) substituted or unsubstituted C1-C6 alkyl,

(8) amino or substituted amino,

(9) substituted or unsubstituted aryl,

(10) substituted or unsubstituted heteroaryl, and

(11) substituted or unsubstituted heterocyclyl;

R⁴ is hydrogen or substituted or unsubstituted C1-C6 alkyl;

R⁵ is hydrogen, alkyl, alkoxy, or halo;

each of R⁶, R⁷, R⁸, and R⁹ are independently selected from the group consisting of hydrogen, alkyl, alkoxy, halo, substituted or unsubstituted aryl, and substituted or unsubstituted heteroaryl; or

a stereoisomer, tautomer, pharmaceutically acceptable salt, or prodrug thereof, and with the proviso that when R^{a} is amino and R⁶, R⁷, R⁸, and R⁹ are hydrogen, then R⁵ is not hydrogen, alkyl, alkoxy, or halo.

In some embodiments, compounds of formula (IIIa) are provided:

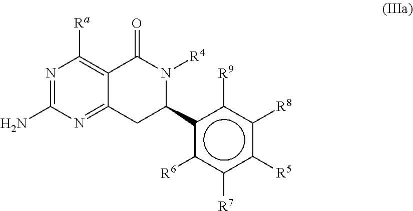

or a tautomer, pharmaceutically acceptable salt, or prodrug thereof wherein R^{a}, R⁴, R⁵, R⁶, R⁷, R⁸, and R⁹ are as previously defined for formula (III) and with the proviso that when R^{a} is amino and R⁶, R⁷, R⁸, and R⁹ are hydrogen, then R⁵ is not hydrogen, alkyl, alkoxy, or halo.

In some embodiments, R^{a} is hydrogen.

In some embodiments, R^{a} is substituted or unsubstituted C1-C6 alkyl.

In some embodiments, R^{a} is C1-C6 alkyl or halo C1-C6 alkyl. In some such embodiments, R^{a} is methyl.

In some embodiments of the invention, R⁴ is selected from the group consisting of hydrogen, benzyl, 1-(4-methoxyphenyl)ethyl, methyl, 3-aminopropyl, and 2-methyl-2-morpholinopropyl. In other embodiments, R is selected from the group consisting of methyl, ethyl, allyl, 3-methyl-butyl, and isobutyl.

In some embodiments, R⁵ is hydrogen or fluoro. In some aspects, R⁵ is fluoro.

In some embodiments, R⁵ is methyl or methoxy.

In some embodiments, R⁷, R⁸, and R⁹ are each hydrogen.

In some embodiments, R is aryl or heteroaryl substituted with one to two substituents selected from the group consisting of halo, alkoxy, alkyl, amino, alkylamino, haloalkyl, and haloalkoxy.

In some embodiments R is selected from the group consisting of substituted aryl and substituted heteroaryl, wherein said aryl and heteroaryl is selected from the group consisting of furanyl, pyrrolyl, phenyl, pyridyl, pyrazinyl, pyrimidinyl, pyridazinyl, pyrazolyl, imidazolyl, triazolyl, indolyl, oxadiazole, thiadiazole, quinolinyl, isoquinolinyl, isoxazolyl, oxazolyl, thiazolyl, and thienyl. In some aspects, the aforementioned groups are substituted with one to two substituents selected from the group consisting of halo, alkoxy, alkyl, amino, alkylamino, haloalkyl, and haloalkoxy.

In other embodiments R⁶ is selected from the group consisting of (2-hydroxy-ethylamino)-pyrazin-2-yl, 1H-pyrazol-4-yl, 1-methyl-1H-pyrazol-4-yl, 1-methyl-1H-pyrazol-4-yl, 2-(5-methyl-pyridin-2-yl)-phenyl, 2,3-difluoro-phenyl, 2,3-dimethoxy-phenyl, 2,4-difluoro-phenyl, 2,4-dimethoxy-phenyl, 2,4-dimethoxy-pyrimidin-5-yl, 2,5-difluoro-phenyl, 2,6-difluoro-phenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-4-yl, 2-difluoro-3-methoxyphenyl, 2-ethyl-phenyl, 2-ethoxy-thiazol-4-yl, 2-fluoro-3-methoxy-phenyl, 2-fluoro-3-methylphenyl, 2-fluoro-4-methyl-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-isoquinolin-4-yl, 2-methoxy-5-trifluoromethyl-phenyl, 2-methoxy-phenyl, 2-methoxy-pyridin-3-yl, 2-methoxy-pyrimidin-4-yl, 2-methoxy-thiazol-4-yl, 2-methyl-phenyl, 2-methyl-pyridin-3-yl, 2-oxo-1,2-dihydro-pyridin-3-yl, 2-phenoxyphenyl, 2-pyridin-3-yl, 2-pyrimidin-5-yl, 2-trifluoromethoxyphenyl, 2-trifluoromethoxy-phenyl, 3,4-dimethoxy-phenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethylphenyl, 4,5-dimethoxy-pyrimidin-2-yl, 4-amino-5-fluor-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-difluoro-2-methyl-phenyl, 4-ethoxy-5-fluoro-pyrimidin-2-yl, 4-ethoxy-pyrimidin-2-yl, 4-ethoxy-pyrimidin-5-yl, 4-ethyl-) H-pyrazol-3-yl, 4-fluoro-2-methoxy-phenyl, 4-fluoro-2-methyl-phenyl, 4-fluorophenyl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-phenyl, 4-methyl-pyridin-2-yl, 4-methyl-pyridin-3-yl, 4-pyrrolidin-1-yl-pyrimidin-2-yl, 5,6-dimethoxy-pyrazin-2-yl, 5-acetyl-thiophen-2-yl, 5-amino-6-ethoxy-pyrazin-2-yl, 5-amino-6-methoxy-3-methyl-pyrazin-2-yl, 5-amino-6-methoxy-pyridin-2-yl, 5-chloro-4-methoxy-pyrimidin-2-yl, 5-chloro-6-methoxy-pyrazin-2-yl, S-dimethylamino-6-methoxy-pyrazin-2-yl, 5-fluoro-2-methoxyphenyl, 5-fluoro-4-methoxy-pyrimidin-2-yl, 5-fluoro-6-methoxy-pyrazin-2 yl, 5-fluoro-pyridin-2-yl, 5-methoxy-pyridin-3-yl, 5-methoxy-thiophen-2-yl, 5-trifluoromethyl-pyrimidin-2-yl, 6-acetyl-pyridin-2-yl, 6-chloro-pyrazin-2-yl, 6-ethoxy-pyrazin-2-yl, 6-ethoxy-pyridin-2-yl, 6-fluoro-pyridin-2-yl, 6-fluoro-pyridin-3-yl, 6-hydroxy-pyridin-2-yl, 6-methoxy-5-methylamino-pyrazin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, 5-amino-6-(2,2,2-trifluoroethoxy)pyrazin-2-yl, and 6-trifluoromethyl-pyridin-2-yl.

### DETAILED DESCRIPTION OF THE INVENTION

The following definitions are provided to better understand the invention.

“Alkyl” or “unsubstituted alkyl” refers to saturated hydrocarbyl groups that do not contain heteroatoms. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. Thus the phrase “alkyl groups” includes primary alkyl groups, secondary alkyl groups, and tertiary alkyl groups. Preferred alkyl groups include straight and branched chain alkyl groups having 1 to 12, 1 to 6, or 1 to 3 carbon atoms.

“Alkylene” or “unsubstituted alkylene” refers to the same residues as noted above for “alkyl,” but having two points of attachment. Exemplary alkylene groups include ethylene (—CH₂CH₂—), propylene (—CH₂CH₂CH₂—), and dimethylpropylene (—CH₂C(CH₃)₂CH₂—).

“Alkenyl” or “unsubstituted alkenyl” refers to straight chain and branched, chain hydrocarbyl radicals having one or more carbon-carbon double bonds and from 2 to about 20 carbon atoms. Preferred alkenyl groups include straight chain and branched alkenyl groups having 2 to 12, or 2 to 6 carbon atoms.

“Alkynyl” or “unsubstituted alkynyl” refers to straight chain and branched chain hydrocarbyl radicals having one or more carbon-carbon triple bonds and from 2 to about 20 carbon atoms. Preferred alkynyl groups include straight chain and branched alkynyl groups having 2 to 12, or 2 to 6 carbon atoms.

“Cycloalkyl” or “unsubstituted cycloalkyl” refers to a mono- or polycyclic alkyl substituent. Representative cycloalkyl groups include cyclopropyl, cyclobutyl, cyclopentyl, cyclohexyl, cycloheptyl, and cyclooctyl. Preferred cycloalkyl groups have 3 to 7 carbon atoms.

“Cycloalkenyl” or “unsubstituted cycloalkenyl” refers to a mono- or polycyclic alkyl substituents having at least one ring carbon-carbon double bond. Preferred cycloalkenyl groups have 5 to 7 carbon atoms and include cyclopentenyl and cyclohexenyl.

“Substituted alkyl” refers to an alkyl group as defined above in which one or more bonds to a carbon(s) or hydrogen(s) are replaced by a bond to non-hydrogen or non-carbon atoms such as, but not limited to, a halogen atom such as F, Cl, Br, and I; an oxygen atom in groups such as hydroxyl groups, alkoxy groups, aryloxy groups, and ester groups; a sulfur atom in groups such as thiol groups, alkyl and aryl sulfide, sulfone, sulfonyl, and sulfoxide groups; a nitrogen atom in groups such as amino, amido, alkylamino, arylamino, alkylarylamino, diarylamino, N-oxides, imides, and enamines. Substituted alkyl groups also include groups in which one or more bonds to a carbon(s) or hydrogen(s) atom is replaced by a higher-order bond (e.g., a double- or triple-bond) to a heteroatom such as oxygen in oxo, carbonyl, carboxyl, and ester groups; or nitrogen in groups such as imines, oximes, hydrazones, and nitriles. Substituted alkyl groups further include alkyl groups in which one or more bonds to a carbon(s) or hydrogen(s) atoms is replaced by a bond to an aryl, heteroaryl, heterocyclyl, cycloalkyl, or cycloalkenyl group. Preferred substituted alkyl groups include, among others, alkyl groups in which one or more bonds to a carbon or hydrogen atom is/are replaced by one or mom bonds to fluoro, chloro, or bromo group. Another preferred substituted alkyl group is the trifluoromethyl group and other alkyl groups that contain the trifluoromethyl group. Other preferred substituted alkyl groups include those in which one or more bonds to a carbon or hydrogen atom is replaced by a bond to an oxygen atom such that the substituted alkyl group contains a hydroxyl, alkoxy, or aryloxy group. Other preferred substituted alkyl groups include alkyl groups that have an amino, or a substituted or unsubstituted alkylamino, arylamino, heterocyclylamino. Still other preferred substituted alkyl groups include those in which one or more bonds to a carbon(s) or hydrogen(s) atoms is replaced by a bond to an aryl, heteroaryl, heterocyclyl, or cycloalkyl group. Examples of substituted alkyl are: —(CH₂)₃NH₂, —(CH₂)₃NH(CH₃), —(CH₂)₃NH(CH₃)₂, —CH₂C(═CH₂)CH₂NH₂, —CH₂C(═O)CH₂NH₂, —CH₂S(═O)₂CH₃, —CH₂OCH₂NH₂, —CH₂CO₂H. Examples of substituents of substituted alkyl are: —CH₂OH, —OH, —OCH₃, —OC₂H₅, —OCF₃, OC(═O)CH₃, —OC(═O)NH₂, —OC(═O)N(CH₃)₂, —CN, —NO₂, —C(═O)CH₃, —CO₂H, —CO₂CH₃, —CONH₂, —NH₂, —N(CH₃)₂, —NHSO₂CH₃, —NHCOCH₃, —NHC(═O)OCH₃, —NHSO—₂CH₃, —SO₂CH₃, —SO₂NH₂, and halo.

“Substituted alkenyl” has the same meaning with respect to unsubstituted alkenyl groups that substituted alkyl groups has with respect to unsubstituted alkyl groups. A substituted alkenyl group includes alkenyl groups in which a non-carbon or non-hydrogen atom is bonded to a carbon double bonded to another carbon and those in which one of the non-carbon or non-hydrogen atoms is bonded to a carbon not involved in a double bond to another carbon.

“Substituted alkynyl” has the same meaning with respect to unsubstituted alkynyl groups that substituted alkyl groups has with respect to unsubstituted alkyl groups. A substituted alkynyl group includes alkynyl groups in which a non-carbon or non-hydrogen atom is bonded to a carbon triple bonded to another carbon and those in which a non-carbon or non-hydrogen atom is bonded to a carbon not involved in a triple bond to another carbon.

“Substituted cycloalkyl” has the same meaning with respect to unsubstituted cycloalkyl groups that substituted alkyl groups has with respect to unsubstituted alkyl groups.

“Substituted cycloalkenyl” has the same meaning with respect to unsubstituted cycloalkenyl groups that substituted alkyl groups has with respect to unsubstituted alkyl groups.

“Aryl” or “unsubstituted aryl” refers to monocyclic and polycyclic aromatic groups that do not contain ring heteroatoms. Such groups can contain from 6 to 14 carbon atoms but preferably 6. Exemplary aryl moieties employed as substituents in compounds of the present invention include phenyl, naphthyl, and the like.

“Aralkyl” or “arylalkyl” refers to an alkyl group substituted with an aryl group as defined above. Typically, aralkyl groups employed in compounds of the present invention have from 1 to 6 carbon atoms incorporated within the alkyl portion of the aralkyl group. Suitable aralkyl groups employed in compounds of the present invention include, for example, benzyl and the like. “Heteroarylalkyl” or “heteroalkyl” refers to an alkyl group substituted with a heteroaryl group as defined above. Typically, heteroarylalkyl groups employed in compounds of the present invention have from 1 to 6 carbon atoms incorporated within the alkyl portion of the aralkyl group. Suitable heteroarylalkyl groups employed in compounds of the present invention include, for example, picolyl and the like.

“Alkoxy” refers to R²⁰O— wherein R²⁰ is C₁-C₇ alkyl or substituted alkyl. In some embodiments, R²⁰ is C₁-C₆ alkyl. Representative examples of alkoxy groups include methoxy, ethoxy, t-butoxy, trifluoromethoxy, and the like.

“Amino” refers herein to the group —NH₂.

“Substituted amino” refers to the group —NR⁶⁰R⁶¹ where R⁶⁰ and R⁶¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, and where R⁶⁰ and R⁶¹ are joined, together with the nitrogen bound thereto to form a heterocyclic or substituted heterocyclic group provided that R⁶⁰ and R⁶¹ are both not hydrogen. When R⁶⁰ is hydrogen and R⁶¹ is alkyl, the substituted amino group is sometimes referred to herein as alkylamino. When R⁶⁰ and R⁶¹ are alkyl, the substituted amino group is sometimes referred to herein as dialkylamino. When referring to a monosubstituted amino, it is meant that either R⁶⁰ and R⁶¹ is hydrogen but not both. When referring to a disubstituted amino, it is meant that neither R⁶⁰ and R⁶¹ is hydrogen. The term “alkylamino” refers herein to the group —NR⁶⁰R⁶¹ where R⁶⁰ is C₁-C₇ alkyl and R⁶⁰ is hydrogen or C₁-C₇ alkyl. The term “dialkylamino” refers to the group —NR⁶⁰R⁶¹ where R⁶⁰ and R⁶¹ are C₁-C₇ alkyl. The term “arylamino” refers herein to the group —NR⁶⁰R⁶¹ where R⁶⁰ is C₅-C₇ aryl and R⁶¹ is hydrogen, C₁-C₇ alkyl, or C₅-C₇ aryl. The term “aralkylamino” refers herein to the group —NR⁶⁰R⁶¹ where R⁶⁰ is aralkyl and R⁶¹ is hydrogen, C₁-C₇ alkyl, C₅-C₇ aryl, or C₅-C₇ aralkyl.

“Amidino” refers to the moieties R⁴⁰—C(═N)—NR⁴¹— (the radical being at the “N¹” nitrogen) and R⁴⁰(NR⁴¹)C═N— (the radical being at the “N²” nitrogen), where R⁴⁰ and R⁴¹ can be hydrogen, C₁-C₇ alkyl, aryl, or C₅-C₇ aralkyl.

“Alkoxyalkyl” refers to the group -alk₁-O-alk₂ where alk₁ is C₁-C₇ alkyl, and alk₂ is C₁-C₇ alkyl. The term “aryloxyalkyl” refers to the group —(C₁-C₇ alkyl)-O—(C₅-C₇ aryl).

“Alkoxyalkylamino” refers herein to the group —NR²⁷-(alkoxyalkyl), where R²⁷ is typically hydrogen, C₅-C₇ aralkyl, or C₁-C₇ alkyl.

“Aminocarbonyl” refers herein to the group —C(O)—NH₂. “Substituted aminocarbonyl” refers herein to the group —C(O)—NR²⁸R²⁹ where R²⁸ is C₁-C₇ alkyl and R²⁹ is hydrogen or C₁-C₇ alkyl. The term “arylaminocarbonyl” refers herein to the group —C(O)—NR³⁰R³¹ where R³⁰ is C₅-C₇ aryl and R³¹ is hydrogen, C₁-C₇ alkyl or C₅-C₇ aryl. “Aralkylaminocarbonyl” refers herein to the group —C(O)—NR³²R³³ where R³² is C₅-C₇ aralkyl and R³³ is hydrogen, C₁-C₇ alkyl, C₅-C₇ aryl, or C₅-C₇ aralkyl.

“Aminosulfonyl” refers herein to the group —S(O)₂—NH₂. “Substituted aminosulfonyl” refers herein to the group —S(O)₂—NR³⁴R³⁵ where R³⁴ is C₁-C₇ alkyl and R³⁵ is hydrogen or C₁-C₇ alkyl. The term “aralkylaminosulfonlyaryl” refers herein to the group —(C₅-C₇ aryl)-S(O)₂—NH-aralkyl.

“Aryloxy” refers to R⁵⁰O— wherein R⁵⁰ is aryl.

“Carbonyl” refers to the divalent group —C(O)—. “Alkylcarbonyl” refers to the group —C(O)alkyl. “Arylcarbonyl” refers to the group —C(O)aryl. Similarly, the term “heteroarylcarbonyl”, “aralkylcarbonyl”, and “heteroalkylcarbonyl” refers to —C(O)—R where R is respectively heteroaryl, aralkyl, and heteroaralkyl.

“Carbonyloxy” refers generally to the group —C(O)—O—. Such groups include esters, —C(O)—O—R³⁶, where R³⁶ is C₁-C₇ alkyl, C₃-C₇ cycloalkyl, aryl, or C₅-C₇ aralkyl. The term “arylcarbonyloxy” refers herein to the group —C(O)—O-(aryl). The term “aralkylcarbonyloxy” refers herein to the group —C(O)—O—(C₅-C₇ aralkyl).

“Cycloalkylalkyl” refers to an alkyl group substituted with a cyloalkyl group as defined above. Typically, cycloalkylalkyl groups have from 1 to 6 carbon atoms incorporated within the alkyl portion of the cycloalkylalkyl group.

“Carbonylamino” refers to the divalent group —NH—C(O)— in which the hydrogen atom of the amide nitrogen of the carbonylamino group can be replaced C₁-C₇ alkyl, aryl, or C₅-C₇ aralkyl group. Carbonylamino groups include moieties such as carbamate esters (—NH—C(O)—O—R²⁸) and amido —NH—C(O)—R²⁸, where R²⁸ is a straight or branched chain C₁-C₇ alkyl, C₃-C₇ cycloalkyl, or aryl or C₅-C₇ aralkyl. The term “alkylcarbonylamino” refers to the group —NH—C(O)—R^{28′} where R^{28′} is alkyl having from 1 to about 7 carbon atoms in its backbone structure. The term “arylcarbonylamino” refers to group —NH—C(O)—R²⁹ where R²⁹ is C₅-C₇ aryl. Similarly, the term “aralkylcarbonylamino” refers to carbonylamino where R²⁹ is C₅-C₇ aralkyl.

“Guanidino” or “guanidyl” refers to moieties derived from guanidine, H₂N—C(═NH)—NH₂. Such moieties include those bonded at the nitrogen atom carrying the formal double bond (the “2”-position of the guanidine, e.g., diaminomethyleneamino, (H₂N)₂C═NH—) and those bonded at either of the nitrogen atoms carrying a formal single bond (the “1-” and/or “3”-positions of the guandine, e.g., H₂N—C(═NH)—NH—). The hydrogen atoms at any of the nitrogens can be replaced with a suitable substituent, such as C₁-C₇ alkyl, aryl, or C₅-C₇ aralkyl.

“Halogen” or “halo” refers to chloro, bromo, fluoro, and iodo groups. The term “haloalkyl” refers to an alkyl radical substituted with one or more halogen atoms. “Haloalkyl” groups include —CF₃. The term “haloalkoxy” refers to an alkoxy radical substituted with one or more halogen atoms. “Haloalkoxy” groups include —OCF₃ and —OCH₂CF₃.

“Hydroxyl” or “hydroxy” refers to the group —OH.

“Heterocyclic” or “unsubstituted heterocyclic group,” “heterocycle” or “unsubstituted heterocycle,” and “heterocyclyl” or “unsubstituted heterocyclyl,” “heterocycloalkyl” or “unsubstituted heterocycloalkyl group,” as used herein refers to any non-aromatic monocyclic or polycyclic ring compounds containing a heteroatom selected from nitrogen, oxygen, or sulfur. Examples include 3- or 4-membered ring containing a heteroatom selected from nitrogen, oxygen, and sulfur or a 5- or 6-membered ring containing from one to three heteroatoms selected from the group consisting of nitrogen, oxygen, or sulfur; wherein the 5-membered ring has 0-1 double bonds and the 6-membered ring has 0-2 double bonds; wherein the nitrogen and sulfur atom maybe optionally oxidized; wherein the nitrogen and sulfur heteroatoms maybe optionally quarternized; and including any bicyclic group in which any of the above heterocyclic rings is fused to a benzene ring or another 5- or 6-membered heterocyclic ring independently defined above provided that the point of attachment is through the heterocyclic ring.

Heterocyclic moieties can be, for example monosubstituted or disubstituted with various substituents independently selected from but not limited to hydroxy, alkoxy, halo, oxo (C═O), alkylimino (R³¹N═, wherein R³¹ is alkyl or alkoxy group), amino, alkylamino, acylaminoalkyl, alkoxy, thioalkoxy, polyalkoxy, alkyl, cycloalkyl or haloalkyl.

The heterocyclic groups may be attached at various positions as shown below as will be apparent to those having skill in the organic and medicinal chemistry arts in conjunction with the disclosure herein.

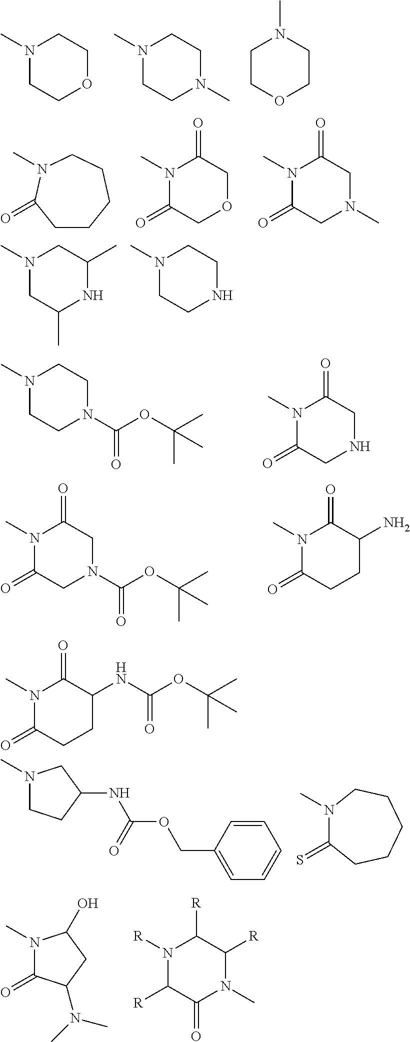

where R is H or a heterocyclic substituent, as described herein.

“Heteroaryl” or “unsubstituted heteroaryl” refers herein to an aromatic group having from 1 to 4 heteroatoms as ring atoms in an aromatic ring with the remainder of the ring atoms being carbon atoms. The term “heteroaryl” includes rings in which nitrogen is the heteroatom as well as partially and fully-saturated rings in which at least one cyclic structure is aromatic, such as, for example, benzodioxozolo (which has a heterocyclic structure fused to a phenyl group, i.e.,

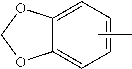

provided that the point of attachment is through the heteroaryl ring. Heteroaryl groups can be further substituted and may be attached at various positions as will be apparent to those having skill in the organic and medicinal chemistry arts in conjunction with the disclosure herein. Representative substituted and unsubstituted heteroaryl groups include, for example, those found in the compounds disclosed in this application and in the examples shown below

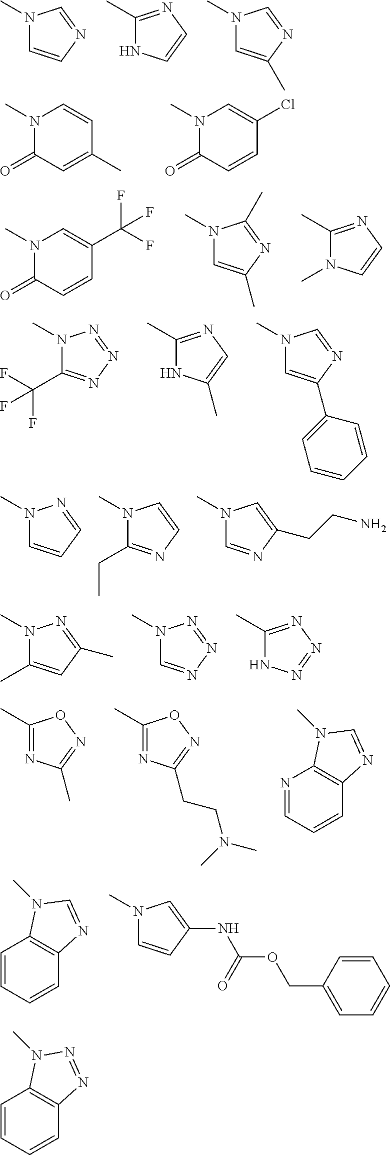

Preferred heterocycles and heteroaryls have 3 to 14 ring atoms and include, for example: diazapinyl, pyrroyl, pyrrolidinyl, pyrazolyl, pyrazolidinyl, imidazoyl, imidazolidinyl, pyridyl, piperidinyl, pyrazinyl, piperazinyl, azetidinyl, pyrimidinyl, pyridazinyl, oxazolyl, oxazolidinyl, isoxazolyl, isoxazolidinyl, morpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, triazolyl, quinoxalinyl, phthalazinyl, naphthpyridinyl, indazolyl, and benzothienyl.

“Heteroarylalkyl” or “heteroaralkyl” refers to an alkyl group substituted with a heteroaryl group as defined above. Typically, heteroarylalkyl groups have from 1 to 6 carbon atoms incorporated within the alkyl portion of the heteroarylalkyl group.

“Imino” refers to the group ═NH.

“Nitro” refers to the group NO₂.

“Sulfonyl” refers herein to the group —SO₂—. “Alkylsulfonyl” refers to a substituted sulfonyl of the structure —SO₂R⁵²— in which R⁵² is C₁-C₇ alkyl. Alkylsulfonyl groups employed in compounds of the present invention are typically alkylsulfonyl groups having from 1 to 6 carbon atoms in its backbone structure. Thus, typical alkylsulfonyl groups employed in compounds of the present invention include, for example, methylsulfonyl (i.e., where R⁵² is methyl), ethylsulfonyl (i.e., where R⁵² is ethyl), propylsulfonyl (i.e., where R⁵² is propyl), and the like. The term “arylsulfonyl” refers herein to the group —SO₂-aryl. The term “heterocyclylsulfonyl” refers herein to the group —SO₂-heterocyclyl. The term “aralkylsulfonyl” refers herein to the group —SO₂-aralkyl. The term “sulfonamido” refers herein to —SO₂NH₂. The term “sulfonamidoalkyl” refers to (alkyl)SO₂NH₂—.

“Thio” or “thiol” refers to the group —SH. “Alkylthio” or “alkylthiol” refers to a thio group substituted with an alkyl group such as, for example, a C₁-C₆ alkyl group.

“Thioamido” refers to the group —C(═S)NH₂.

“Optionally substituted” refers to the optional replacement of hydrogen with a monovalent or divalent radical. “Substituted” refers to the replacement of hydrogen with a monovalent or divalent radical. Unless indicated otherwise, suitable substitution groups include, for example, hydroxyl, alkoxy, nitro, amino, imino, cyano, halo, thio, sulfonyl, thioamido, amidino, oxo, oxamidino, methoxamidino, guanidino, sulfonamido, carboxyl, formyl, alkyl, haloalkyl, alkylamino, haloalkylamino, alkoxy, haloalkoxy, alkoxy-alkyl, alkylcarbonyl, aminocarbonyl, arylcarbonyl, aralkylcarbonyl, heteroarylcarbonyl, heteroaralkyl-carbonyl, alkylthio, aminoalkyl, cyanoalkyl, aryl and the like. Other suitable substitution groups include those substituents indicated for substituted alkyl. Examples of various suitable substitution groups are also found in reference to the compounds disclosed throughout this application.

The substitution group can itself be substituted. The group substituted onto the substitution group can be carboxyl, halo, nitro, amino, cyano, hydroxyl, alkyl, alkoxy, aminocarbonyl, —SR⁴², thioamido, —SO₃H, —SO₂R⁴², or cycloalkyl, where R⁴² is typically hydrogen, hydroxyl or alkyl.

When the substituted substituent includes a straight chain group, the substitution can occur either within the chain (e.g., 2-hydroxypropyl, 2-aminobutyl, and the like) or at the chain terminus (e.g., 2-hydroxyethyl, 3-cyanopropyl, and the like). Substituted substituents can be straight chain, branched or cyclic arrangements of covalently bonded carbon or heteroatoms.

Unless indicated otherwise, the nomenclature of substituents that are not explicitly defined herein are arrived at by naming the terminal portion of the functionality followed by the adjacent functionality toward the point of attachment. For example, the substituent “alkoxyheteroaryl” refers to the group (alkoxy)-(heteroaryl)-.

Preferred compounds of Formula (I) used in this invention have a total molecular weight less than 1000 Daltons, preferably less than 750 Daltons. Compounds of Formula (I) typically have a minimum molecular weight of at least 150 Daltons. Preferred compounds of Formula (I) have a molecular weight between 150 and 750 Daltons, and in more preferred embodiments, have a molecular weight between 200 and 500 Daltons. Other embodiments of the invention include the use of compounds of Formula (I) with a molecular weight between 300 and 450 Daltons. In another aspect of the invention compounds of Formula (I) used in the invention have a molecular weight between 350 and 400 Daltons.

Similarly, it is understood that the above definitions are not intended to include impermissible substitution patterns (e.g., methyl substituted with 5 fluoro groups). Such impermissible substitution patterns are well known to the skilled artisan.

“Carboxy-protecting group” refers to a carbonyl group which has been esterified with one of the commonly used carboxylic acid protecting ester groups employed to block or protect the carboxylic acid function while reactions involving other functional sites of the compound are carried out. In addition, a carboxy protecting group can be attached to a solid support whereby the compound remains connected to the solid support as the carboxylate until cleaved by hydrolytic methods to release the corresponding free acid. Representative carboxy-protecting groups include, for example, alkyl esters, secondary amides and the like.

Certain of the compounds according to Formula (I) comprise asymmetrically substituted carbon atoms. Such asymmetrically substituted carbon atoms can result in the compounds of the invention comprising mixtures of stereoisomers at a particular asymmetrically substituted carbon atom or a single stereoisomer. As a result, racemic mixtures, mixtures of enantiomers, as well as enantiomers of the compounds of the invention are included in the present invention. The terms “S” and “R” configuration, as used herein, are as defined by the IUPAC 1974 “Recommendations for Section E, Fundamental Stereochemistry,” Pure Appl. Chem. 45:13-30, 1976. The terms α and β are employed for ring positions of cyclic compounds. The α-side of the reference plane is that side on which the preferred substituent lies at the lower numbered position. Those substituents lying on the opposite side of the reference plane are assigned β descriptor. It should be noted that this usage differs from that for cyclic stereoparents, in which “α” means “below the plane” and denotes absolute configuration. The terms α and β configuration, as used herein, are as defined by the “Chemical Abstracts Index Guide,” Appendix IV, paragraph 203, 1987.

As used herein, the term “pharmaceutically acceptable salts” refers to the nontoxic acid or alkaline earth metal salts of compounds of the invention. These salts can be prepared in situ during the final isolation and purification of the compounds, or by separately reacting the base or acid functions with a suitable organic or inorganic acid or base, respectively. Representative salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, persulfate, 3-phenylproionate, picrate, pivalate, propionate, succinate, sulfate, tartrate, thiocyanate, p-toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, sulfuric acid and phosphoric acid and such organic acids as oxalic acid, maleic acid, methanesulfonic acid, succinic acid and citric acid. Basic addition salts can be prepared in situ during the final isolation and purification of the compound, or separately by reacting carboxylic acid moieties with a suitable base such as the hydroxide, carbonate or bicarbonate of a pharmaceutically acceptable metal cation or with ammonia, or an organic primary, secondary or tertiary amine. Pharmaceutically acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, and the like.

The term “pharmaceutically acceptable prodrugs” as used herein refers to those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood, such as an ester prodrug. A thorough discussion is provided in Higuchi, T., and V. Stella, “Pro-drugs as Novel Delivery Systems,” A.C.S. Symposium Series 14, and in “Bioreversible Carriers in Drug Design,” in Edward B. Roche (ed.), American Pharmaceutical Association, Pergamon Press, 1987, both of which are incorporated herein by reference.

In embodiments, a pharmaceutical composition according to the invention comprises a first pharmaceutical component and a second pharmaceutical component in a pharmaceutically acceptable carrier. The first component is a compound according to Formula (I)

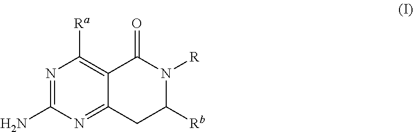

or a stereoisomer, tautomer, pharmaceutically acceptable salt, or prodrug thereof, wherein

R^{a} is selected from the group consisting of

(1) hydrogen,

(2) halogen,

(3) hydroxyl,

(4) C₁-C₆ alkoxy,

(5) thiol,

(6) C₁-C₆ alkylthiol,

(7) substituted or unsubstituted C1-C6 alkyl,

(8) amino or substituted amino,

(9) substituted or unsubstituted aryl,

(10) substituted or unsubstituted heteroaryl, and

(11) substituted or unsubstituted heterocyclyl;

R is selected from the group consisting of

(1) hydrogen,

(2) substituted or unsubstituted C1-C6 alkyl,

(3) substituted or unsubstituted C2-C6 alkenyl,

(4) substituted or unsubstituted C2-C6 alkynyl,

(5) substituted or unsubstituted C3-C7 cycloalkyl,

) (6) substituted or unsubstituted C5-C7 cycloalkenyl,

(7) substituted or unsubstituted aryl,

(8) substituted or unsubstituted heteroaryl, and

(9) substituted or unsubstituted heterocyclyl;

R^{b} is selected from the group consisting of

(1) substituted or unsubstituted C3-C7 cycloalkyl,

(2) substituted or unsubstituted C5-C7 cycloalkenyl,

(3) substituted or unsubstituted aryl,

(4) substituted or unsubstituted heteroaryl, and

(5) substituted or unsubstituted heterocyclyl; and

with the proviso that when R^{a} is amino, then R^{b} is not phenyl, 4-alkyl-phenyl, 4-alkoxy-phenyl, or 4-halo-phenyl.

The second component is a HER2 inhibitor.

Preferably, the first component is an Hsp 90 inhibitor.

In particular embodiments, the first component is a HSP90 inhibitor compound according to formula (Ia)

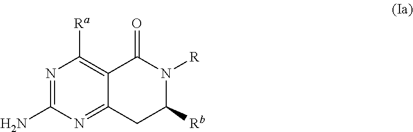

or a tautomer, pharmaceutically acceptable salt, or prodrug thereof, wherein R, R^{a}, and R^{b} are as previously defined for Formula (I) and with the proviso that when R^{a} is amino, then R^{b} is not phenyl, 4-alkyl-phenyl, 4-alkoxy-phenyl, or 4-halo-phenyl.

In some embodiments of the compounds of Formula (I) or (Ia), R^{a} is hydrogen.

In other embodiments, R^{a} is substituted or unsubstituted C1-C6 alkyl.

In some embodiments, R^{a} is C1-C6 alkyl or halo C1-C6 alkyl. In some such embodiments, R^{a} is methyl.

In some embodiments, R^{b} is aryl or heteroaryl. In some such embodiments, R^{b} is selected from the group consisting of phenyl, pyridyl, pyrimidinyl, pyrazinyl, indolyl, thiazolyl, and thienyl, each of which can be substituted or unsubstituted. In some aspects, the invention provides compounds wherein the aforementioned R^{b} groups are substituted with substituted or unsubstituted aryl, or substituted or unsubstituted heteroaryl. In other aspects the R^{b} groups are substituted with halo. In still other aspects the R^{b} groups are substituted with fluoro. In still other aspects, the R^{b} groups are substituted with alkyl, haloalkyl, alkoxy, and haloalkoxy. In some aspects, the R^{b} groups are substituted with methyl. In other aspects, the R^{b} groups are substituted with methoxy.

In other embodiments, R^{b} is selected from the group consisting of substituted aryl, substituted heterocyclyl, substituted heteroaryl, substituted C3-C7 cycloalkyl, and substituted C5-C7 cycloalkenyl, wherein said aryl, heterocyclyl, heteroaryl, C3-C7 cycloalkyl, and C5-C7 cycloalkenyl is selected from the group consisting of pyrrolyl, phenyl, pyridyl, pyrazinyl, pyrimidinyl, pyridazinyl, pyrazolyl, imidazolyl, triazolyl, indolyl, oxadiazole, thiadiazole, furanyl, quinolinyl, isoquinolinyl, isoxazolyl, oxazolyl, thiazolyl, morpholino, piperidinyl, pyrrolidinyl, thienyl, cyclohexyl, cyclopentyl, cyclohexenyl, and cyclopentenyl. In some aspects, the aforementioned groups are substituted with one to two substituents selected from the group consisting of halo, alkoxy, alkyl, amino, alkylamino, haloalkyl, and haloalkoxy.

In some embodiments, R is selected from the group consisting of hydrogen, unsubstituted alkyl, and substituted alkyl. In some such embodiments, R is selected from the group consisting of methyl, ethyl, allyl, 3-methyl-butyl, and isobutyl. In other embodiments, R is selected from the group consisting of hydrogen, benzyl, 1-(4-methoxyphenyl)ethyl, methyl, 3-aminopropyl, and 2-methyl-2-morpholinopropyl. In still other embodiment, R is hydrogen.

In another embodiment, the 2-amino-7,8-dihydro-6H-pyrido[4,3-d]pyrimidin-5-one compounds have the formula (II):

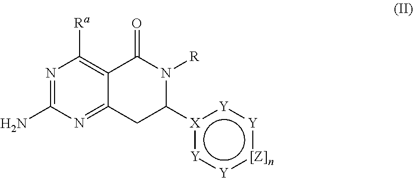

or a stereoisomer, tautomer, pharmaceutically acceptable salt, or prodrug thereof, wherein

n is 0 or 1,

wherein R is selected from the group consisting of

(1) hydrogen,

(2) halogen,

(3) hydroxyl,

(4) C₁-C₆ alkoxy,

(5) thiol,

(6) C1-C₆ alkylthiol,

(7) substituted or unsubstituted C1-C6 alkyl,

(8) amino or substituted amino,

(9) substituted or unsubstituted aryl,

(10) substituted or unsubstituted heteroaryl, and

(11) substituted or unsubstituted heterocyclyl;

wherein R is selected from the group consisting of

(1) hydrogen,

(2) substituted or unsubstituted C1-C6 alkyl,

(3) substituted or unsubstituted C2-C6 alkenyl,

(4) substituted or unsubstituted C2-C6 alkynyl,

(5) substituted or unsubstituted C3-C7 cycloalkyl,

(6) substituted or unsubstituted C5-C7 cycloalkenyl,

(7) substituted or unsubstituted aryl,

(8) substituted or unsubstituted heteroaryl, and

(9) substituted or unsubstituted heterocyclyl,

wherein when n is 1, X is C, Y is at each position independently selected from CQ¹ and N, and Z is selected from CR² and N with the proviso that no more than 3 Y and Z groups are N, and

wherein when n is 0, X is C or N, Y is at each position independently selected from CQ¹, N, NQ², O, and S with the proviso that no more than 4 X and Y groups are N and NQ² and no more than 1 Y group is S or O;

wherein Q¹ is at each position independently selected from the group consisting of

(1) hydrogen,

(2) halogen,

(3) substituted or unsubstituted C1-C6 alkyl,

(4) substituted or unsubstituted C2-C6 alkenyl,

(5) substituted or unsubstituted C2-C6 alkynyl,

(6) substituted or unsubstituted C3-C7 cycloalkyl,

(7) substituted or unsubstituted C5-C7 cycloalkenyl,

(8) substituted or unsubstituted aryl,

(9) substituted or unsubstituted heteroaryl,

(10) substituted or unsubstituted heterocyclyl,

(11) substituted or unsubstituted amino,

(12) —OR³ or —SR³

(13) —C(O)R³, —CO₂R³, —C(O)N(R³)₂, —S(O)R³, —SO₂R₃, or —SO₂N(R³)₂,

(14) —OC(O)R³, —N(R³)C(O)R³, or —N(R³)SO₂R₃,

(15) —CN, and

(16) —NO₂;

wherein Q² is at each position independently selected from the group consisting of

(1) hydrogen,

(3) substituted or unsubstituted C1-C6 alkyl,

(4) substituted or unsubstituted C2-C6 alkenyl,

(5) substituted or unsubstituted C2-C6 alkynyl,

(6) substituted or unsubstituted C3-C7 cycloalkyl,

(7) substituted or unsubstituted C5-C7 cycloalkenyl,

(8) substituted or unsubstituted aryl,

(9) substituted or unsubstituted heteroaryl, and

(10) substituted or unsubstituted heterocyclyl;

wherein R² is selected from the group consisting of

(1) hydrogen,

(2) halogen,

(3) substituted or unsubstituted C1-C3 alkyl, and

(4) —OR³, —SR³, or —NHR³;

wherein R³ is at each position independently selected from the group consisting of

(1) hydrogen,

(2) substituted or unsubstituted C1-C6 alkyl,

(3) substituted or unsubstituted C2-C6 alkenyl,

(4) substituted or unsubstituted C2-C6 alkynyl,

(5) substituted or unsubstituted C3-C7 cycloalkyl,

(6) substituted or unsubstituted C5-C7 cycloalkenyl,

(7) substituted or unsubstituted aryl,

(8) substituted or unsubstituted heteroaryl, and

(9) substituted or unsubstituted heterocyclyl,

with the proviso that when R^{a} is amino, then X, Y, Z, and n together do not form a phenyl, 4-alkyl-phenyl, 4-alkoxy-phenyl, or 4-halo-phenyl group.

In other embodiments, the first pharmaceutical component of the invention is described according to formula (IIa):

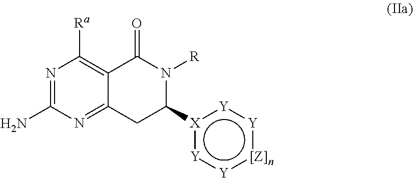

or a tautomer, pharmaceutically acceptable salt, or prodrug thereof, wherein R^{a}, R, X, Y, Z, and n are previously defined for formula (II) and with the proviso that when R^{a} is amino, then X, Y, Z, and n together do not form a phenyl, 4-alkyl-phenyl, 4-alkoxy-phenyl, or 4-halo-phenyl group.

In some embodiments when n is 0, X is C, and Y adjacent to X is not O.

In some embodiments of the compounds of formula (II) or (Ha), R is hydrogen.

In other embodiments, R^{a} is substituted or unsubstituted C1-C6 alkyl.

In some embodiments, R^{a} is C1-C6 alkyl or halo C1-C6 alkyl. In some such embodiments, R^{a} is methyl.

For the compounds of Formula (I), (Ia), (II), or (IIa), representative substituted alkyl groups include arylalkyl, heteroarylalkyl, cycloalkylalkyl, heterocyclylalkyl, aminoalkyl, alkylaminoalkyl, dialkylaminoalkyl, and sulfonamidoalkyl groups.

Representative aryl groups include phenyl groups.

Representative heteroaryl groups include pyridyl, pyrazinyl, pyrimidinyl, pyridazinyl, pyrazolyl, indolyl, quinolinyl, isoquinolinyl, furanyl, oxazolyl, thiazolyl, and thienyl groups.

In one embodiment, one of Q¹ or Q² is selected from the group consisting of substituted and unsubstituted phenyl, substituted and unsubstituted pyridyl, substituted and unsubstituted pyrimidinyl, substituted and unsubstituted pyrazinyl, substituted and unsubstituted indolyl, substituted and unsubstituted thiazolyl, and substituted and unsubstituted thienyl.

In one embodiment, one of Q¹ or Q² is selected from the group consisting of piperidinyl, morpholinyl, pyrrolidinonyl, and benzyl amino.

In one embodiment, one of Q¹ or Q² is selected from the group consisting of cyclohexyl and cyclopentyl.

In one embodiment, one of Q¹ or Q² is selected from the group consisting of cyclohexenyl and cyclopentenyl.

In one embodiment, one of Q¹ or Q² is selected from the group consisting of substituted aryl, substituted heterocyclyl, substituted heteroaryl, substituted C3-C7 cycloalkyl, and substituted C5-C7 cycloalkenyl, wherein said aryl, heterocyclyl, heteroaryl, C3-C7 cycloalkyl, and C5-C7 cycloalkenyl is selected from the group consisting of pyrrolyl, phenyl, pyridyl, pyrazinyl, pyrimidinyl, pyridazinyl, pyrazolyl, imidazolyl, triazolyl, indolyl, oxadiazole, thiadiazole, furanyl, quinolinyl, isoquinolinyl, isoxazolyl, oxazolyl, thiazolyl, morpholino, piperidinyl, pyrrolidinyl, thienyl, cyclohexyl, cyclopentyl, cyclohexenyl, and cyclopentenyl.

In some aspects, the aforementioned groups are substituted with one to two substituents selected from the group consisting of halo, alkoxy, alkyl, amino, alkylamino, haloalkyl, and haloalkoxy.

In one embodiment, one of Q¹ or Q² is selected from substituted and unsubstituted pyridyl, substituted and unsubstituted pyrazinyl, substituted and unsubstituted phenyl, substituted and unsubstituted isoquinolinyl, substituted and unsubstituted pyrimidinyl, substituted and unsubstituted pyrazolyl, and substituted and unsubstituted furanyl. In some aspects, the aforementioned groups are substituted with one to two substituents selected from the group consisting of halo, alkoxy, alkyl, amino, alkylamino, haloalkyl, and haloalkoxy.

In other embodiments one of Q¹ or Q² is selected from the group consisting of (2-hydroxy-ethylamino)-pyrazin-2-yl, 1H-pyrazol-4-yl, 1-methyl-1H-pyrazol-4-yl, 1-methyl-1H-pyrazol-4-yl, 2-(5-methyl-pyridin-2-yl)-phenyl, 2,3-difluoro-phenyl, 2,3-dimethoxy-phenyl, 2,4-difluoro-phenyl, 2,4-dimethoxy-phenyl, 2,4-dimethoxy-pyrimidin-5-yl, 2,5-difluoro-phenyl, 2,6-difluoro-phenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-4-yl, 2-difluoro-3-methoxyphenyl, 2-ethyl-phenyl, 2-ethoxy-thiazol-4-yl, 2-fluoro-3-methoxy-phenyl, 2-fluoro-3-methylphenyl, 2-fluoro-4-methyl-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-isoquinolin-4-yl, 2-methoxy-5-trifluoromethyl-phenyl, 2-methoxy-phenyl, 2-methoxy-pyridin-3-yl, 2-methoxy-pyrimidin-4-yl, 2-methoxy-thiazol-4-yl, 2-methyl-phenyl, 2-methyl-pyridin-3-yl, 2-oxo-1,2-dihydro-pyridin-3-yl, 2-phenoxyphenyl, 2-pyridin-3-yl, 2-pyrimidin-5-yl, 2-trifluoromethoxyphenyl, 2-trifluoromethoxy-phenyl, 3,4-dimethoxy-phenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethylphenyl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-difluoro-2-methyl-phenyl, 4-ethoxy-5-fluoro-pyrimidin-2-yl, 4-ethoxy-pyrimidin-2-yl, 4-ethoxy-pyrimidin-5-yl, 4-ethyl-1H-pyrazol-3-yl, 4-fluoro-2-methoxy-phenyl, 4-fluoro-2-methyl-phenyl, 4-fluorophenyl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-phenyl, 4-methyl-pyridin-2-yl, 4-methyl-pyridin-3-yl, 4-pyrrolidin-1-yl-pyrimidin-2-yl, 5,6-dimethoxy-pyrazin-2-yl, 5-acetyl-thiophen-2-yl, 5-amino-6-ethoxy-pyrazin-2-yl, 5-amino-6-methoxy-3-methyl-pyrazin-2-yl, 5-amino-6-methoxy-pyridin-2-yl, 5-chloro-4-methoxy-pyrimidin-2-yl, 5-chloro-6-methoxy-pyrazin-2-yl, 5-dimethylamino-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-methoxy-thiophen-2-yl, 5-trifluoromethyl-pyrimidin-2-yl, 6-acetyl-pyridin-2-yl, 6-chloro-pyrazin-2-yl, 6-ethoxy-pyrazin-2-yl, 6-ethoxy-pyridin-2-yl, 6-fluoro-pyridin-2-yl, 6-fluoro-pyridin-3-yl, 6-hydroxy-pyridin-2-yl, 6-methoxy-5-methylamino-pyrazin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, 5-amino-6-(2,2,2-trifluoroethoxy)pyrazin-2-yl, and 6-trifluoromethyl-pyridin-2-yl.

In one embodiment Q¹ is halo.

In one embodiment Qt is alkyl. In some aspects, Q¹ is methyl.

In one embodiment, R² is selected from hydrogen and fluoro. In some aspects, R² is fluoro.

In one embodiment, R² is selected from alkyl. In some aspects, R² is methyl.

In one embodiment, R² is selected from alkoxy. In some aspects, R² is methoxy.

In one embodiment Q¹ is OR³.

In one embodiment, R³ is selected from the group consisting of methyl, ethyl, isopropyl, cyclopentyl, and cyclohexyl.

In one embodiment, R³ is selected from substituted and unsubstituted phenyl, substituted and unsubstituted thiazolyl, substituted and unsubstituted pyridyl, substituted and unsubstituted pyrazinyl, and substituted and unsubstituted pyrimidinyl.

In one embodiment, R³ is selected from the group consisting of 2-aminoethyl, 2-piperidinylethyl, 2-piperazinylethyl, 2-morpholinylethyl, and 2-(N-methylpiperazinyl)ethyl.

In some embodiments, R is selected from the group consisting of hydrogen, unsubstituted alkyl, and substituted alkyl. In some such embodiments, R is selected from the group consisting of methyl, ethyl, allyl, 3-methyl-butyl, and isobutyl. In other embodiments, R is selected from the group consisting of hydrogen, benzyl, 1-(4-methoxyphenyl)ethyl, methyl, 3-aminopropyl, and 2-methyl-2-morpholinopropyl.

In another embodiment of the invention, compounds of formula (III) are provided as the first component, in combination with a HER2 inhibitor as the second component:

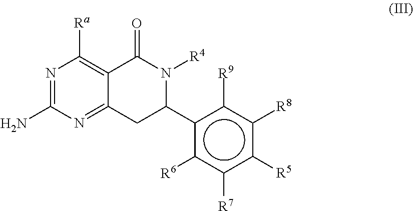

or a stereoisomer, tautomer, pharmaceutically acceptable salt, or prodrug thereof, wherein

wherein R is selected from the group consisting of

(1) hydrogen,

(2) halogen,

(3) hydroxyl,

(4) C₁-C₆ alkoxy,

(5) thiol,

(6) C₁-C₅ alkylthiol,

(7) substituted or unsubstituted C1-C6 alkyl,

(8) amino or substituted amino,

(9) substituted or unsubstituted aryl,

(10) substituted or unsubstituted heteroaryl, and

(11) substituted or unsubstituted heterocyclyl;

R⁴ is hydrogen or substituted or unsubstituted C1-C6 alkyl;

R⁵ is hydrogen, alkyl, alkoxy, or halo;

each of R⁶, R⁷, R⁸, and R⁹ are independently selected from the group consisting of hydrogen, alkyl, alkoxy, halo, substituted or unsubstituted aryl, and substituted or unsubstituted heteroaryl; or

a stereoisomer, tautomer, pharmaceutically acceptable salt, or prodrug thereof, and with the proviso that when R^{a} is amino and R⁶, R⁷, R⁸, and R⁹ are hydrogen, then R⁵ is not hydrogen, alkyl, alkoxy, or halo.

In some embodiments, compounds of formula (ma) are provided as the first component:

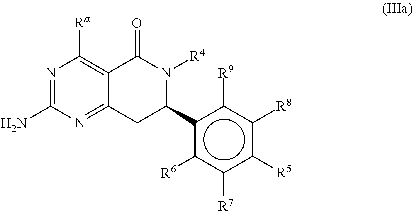

or a tautomer, pharmaceutically acceptable salt, or prodrug thereof, wherein R^{a}, R⁴, R⁵, R⁶, R⁷, R⁸, and R⁹ are as previously defined for formula (III) and with the proviso that when R^{a} is amino and R⁶, R⁷, R⁸, and R⁹ are hydrogen, then R⁵ is not hydrogen, alkyl, alkoxy, or halo.

In some embodiments, R^{a} is hydrogen.

In some embodiments, R^{a} is substituted or unsubstituted C1-C6 alkyl.

In some embodiments, R^{a} is C1-C6 alkyl or halo C1-C6 alkyl. In some such embodiments, R^{a} is methyl.

In some embodiments of the invention, R⁴ is selected from the group consisting of hydrogen, benzyl, 1-(4-methoxyphenyl)ethyl, methyl, 3-aminopropyl, and 2-methyl-2-morpholinopropyl. In other embodiments, R is selected from the group consisting of methyl, ethyl, allyl, 3-methyl-butyl, and isobutyl.

In some embodiments, R⁵ is hydrogen or fluoro. In some aspects, R⁵ is fluoro.

In some embodiments, R⁵ is methyl or methoxy.

In some embodiments, R⁷, R⁸, and R⁹ are each hydrogen.

In some embodiments, R⁶ is aryl or heteroaryl substituted with one to two substituents selected from the group consisting of halo, alkoxy, alkyl, amino, alkylamino, haloalkyl, and haloalkoxy.

In some embodiments R⁶ is selected from the group consisting of substituted aryl and substituted heteroaryl, wherein said aryl and heteroaryl is selected from the group consisting of furanyl, pyrrolyl, phenyl, pyridyl, pyrazinyl, pyrimidinyl, pyridazinyl, pyrazolyl, imidazolyl, triazolyl, indolyl, oxadiazole, thiadiazole, quinolinyl, isoquinolinyl, isoxazolyl, oxazolyl, thiazolyl, and thienyl. In some aspects, the aforementioned groups are substituted with one to two substituents selected from the group consisting of halo, alkoxy, alkyl, amino, alkylamino, haloalkyl, and haloalkoxy.

In other embodiments R⁶ is selected from the group consisting of (2-hydroxy-ethylamino)-pyrazin-2-yl, 1H-pyrazol-4-yl, 1-methyl-1H-pyrazol-4-yl, 1-methyl-1H-pyrazol-4-yl, 2-(5-methyl-pyridin-2-yl)-phenyl, 2,3-difluoro-phenyl, 2,3-dimethoxy-phenyl, 2,4-difluoro-phenyl, 2,4-dimethoxy-phenyl, 2,4-dimethoxy-pyrimidin-5-yl, 2,5-difluoro-phenyl, 2,6-difluoro-phenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-4-yl, 2-difluoro-3-methoxyphenyl, 2-ethyl-phenyl, 2-ethoxy-thiazol-4-yl, 2-fluoro-3-methoxy-phenyl, 2-fluoro-3-methylphenyl, 2-fluoro-4-methyl-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-isoquinolin-4-yl, 2-methoxy-5-trifluoromethyl-phenyl, 2-methoxy-phenyl, 2-methoxy-pyridin-3-yl, 2-methoxy-pyrimidin-4-yl, 2-methoxy-thiazol-4-yl, 2-methyl-phenyl, 2-methyl-pyridin-3-yl, 2-oxo-1,2-dihydro-pyridin-3-yl, 2-phenoxyphenyl, 2-pyridin-3-yl, 2-pyrimidin-5-yl, 2-trifluoromethoxyphenyl, 2-trifluoromethoxy-phenyl, 3,4-dimethoxy-phenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethylphenyl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-difluoro-2-methyl-phenyl, 4-ethoxy-5-fluoro-pyrimidin-2-yl, 4-ethoxy-pyrimidin-2-yl, 4-ethoxy-pyrimidin-5-yl, 4-ethyl-1H-pyrazol-3-yl, 4-fluoro-2-methoxy-phenyl, 4-fluoro-2-methyl-phenyl, 4-fluorophenyl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-phenyl, 4-methyl-pyridin-2-yl, 4-methyl-pyridin-3-yl, 4-pyrrolidin-1-yl-pyrimidin-2-yl, 5,6-dimethoxy-pyrazin-2-yl, 5-acetyl-thiophen-2-yl, 5-amino-6-ethoxy-pyrazin-2-yl, 5-amino-6-methoxy-3-methyl-pyrazin-2-yl, 5-amino-6-methoxy-pyridin-2-yl, 5-chloro-4-methoxy-pyrimidin-2-yl, 5-chloro-6-methoxy-pyrazin-2-yl, 5-dimethylamino-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-methoxy-thiophen-2-yl, 5-trifluoromethyl-pyrimidin-2-yl, 6-acetyl-pyridin-2-yl, 6-chloro-pyrazin-2-yl, 6-ethoxy-pyrazin-2-yl, 6-ethoxy-pyridin-2-yl, 6-fluoro-pyridin-2-yl, 6-fluoro-pyridin-3-yl, 6-hydroxy-pyridin-2-yl, 6-methoxy-5-methylamino-pyrazin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, 5-amino-6-(2,2,2-trifluoroethoxy)pyrazin-2-yl, and 6-trifluoromethyl-pyridin-2-yl.

The first component and the second component may be provided in a pharmaceutically acceptable carrier to form a pharmaceutical composition.

In another embodiment of the invention, compounds of formula (IV) are provided as the first component:

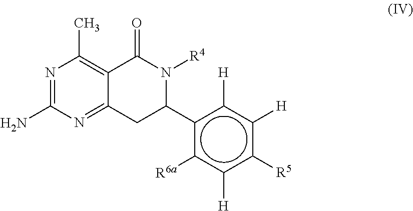

or a stereoisomer, tautomer, pharmaceutically acceptable salt, or prodrug thereof, wherein

R⁴ is hydrogen or substituted or unsubstituted C1-C6 alkyl,

R⁵ is hydrogen or halo,

R^{6a} is selected from the group consisting of halo, substituted or unsubstituted aryl, and substituted or unsubstituted heteroaryl.

In some embodiments compounds of formula (IVa) are provided as the first component:

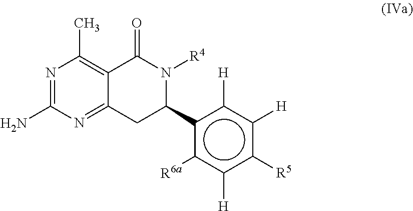

or a tautomer, pharmaceutically acceptable salt, or prodrug thereof, wherein

R⁴, R⁵, and R^{6a} are as previously defined for formula (IV).

In some embodiments of the compounds of formula (IV) or (IVa), R⁴ is selected from the group consisting of hydrogen, benzyl, 1-(4-methoxyphenyl)ethyl, methyl, 3-aminopropyl, and 2-methyl-2-morpholinopropyl. In other embodiments, R is selected from the group consisting of methyl, ethyl, allyl, 3-methyl-butyl, and isobutyl.

In some embodiments, R⁵ is hydrogen or fluoro. In some aspects R⁵ is fluoro.

In some aspects, R^{6a} is aryl or heteroaryl substituted with one to two substituents selected from the group consisting of halo, alkoxy, alkyl, amino, alkylamino, haloalkyl, and haloalkoxy.

In some embodiments R^{6a} is selected from the group consisting of substituted aryl and substituted heteroaryl, wherein said aryl and heteroaryl is selected from the group consisting of furanyl, pyrrolyl, phenyl, pyridyl, pyrazinyl, pyrimidinyl, pyridazinyl, pyrazolyl, imidazolyl, triazolyl, indolyl, oxadiazole, thiadiazole, quinolinyl, isoquinolinyl, isoxazolyl, oxazolyl, thiazolyl, and thienyl. In some aspects, the aforementioned groups are substituted with one to two substituents selected from the group consisting of halo, alkoxy, alkyl, amino, alkylamino, haloalkyl, and haloalkoxy.

In some embodiments, R^{6a} is selected from the group consisting of (2-hydroxy-ethylamino)-pyrazin-2-yl, 1H-pyrazol-4-yl, 1-methyl-1H-pyrazol-4-yl, 1-methyl-1H-pyrazol-4-yl, 2-(5-methyl-pyridin-2-yl)-phenyl, 2,3-difluoro-phenyl, 2,3-dimethoxy-phenyl, 2,4-difluoro-phenyl, 2,4-dimethoxy-phenyl, 2,4-dimethoxy-pyrimidin-5-yl, 2,5-difluoro-phenyl, 2,6-difluoro-phenyl, 2,6-dimethyl-pyridin-3-yl, 2-acetamidophenyl, 2-aminocarbonylphenyl, 2-amino-pyrimidin-5-yl, 2-chloro-4-methoxy-pyrimidin-5-yl, 2-chloro-5-fluoro-pyridin-3-yl, 2-chloro-phenyl, 2-chloro-pyridin-3-yl, 2-chloro-pyridin-4-yl, 2-difluoro-3-methoxyphenyl, 2-ethyl-phenyl, 2-ethoxy-thiazol-4-yl, 2-fluoro-3-methoxy-phenyl, 2-fluoro-3-methylphenyl, 2-fluoro-4-methyl-phenyl, 2-fluoro-5-methoxy-phenyl, 2-fluoro-5-methylphenyl, 2-fluorophenyl, 2-fluoro-pyridin-3-yl, 2-hydroxymethyl-3-methoxyphenyl, 2-hydroxymethylphenyl, 2-isoquinolin-4-yl, 2-methoxy-5-trifluoromethyl-phenyl, 2-methoxy-phenyl, 2-methoxy-pyridin-3-yl, 2-methoxy-pyrimidin-4-yl, 2-methoxy-thiazol-4-yl, 2-methyl-phenyl, 2-methyl-pyridin-3-yl, 2-oxo-1,2-dihydro-pyridin-3-yl, 2-phenoxyphenyl, 2-pyridin-3-yl, 2-pyrimidin-5-yl, 2-trifluoromethoxyphenyl, 2-trifluoromethoxy-phenyl, 3,4-dimethoxy-phenyl, 3,5-dimethyl-isoxazol-4-yl, 3,6-dimethyl-pyrazin-2-yl, 3-acetamidophenyl, 3-aminocarbonylphenyl, 3-bromo-phenyl, 3-chloro-pyrazin-2-yl, 3-cyanophenyl, 3-dimethylaminophenyl, 3-ethoxy-phenyl, 3-ethyl-4-methyl-phenyl, 3-ethynyl-phenyl, 3-fluoro-6-methoxy-pyridin-2-yl, 3-fluorophenyl, 3-fluoro-pyrazin-2-yl, 3-methanesulfonamidophenyl, 3-methoxycarbonylphenyl, 3-methoxyphenyl, 3-methoxy-pyrazin-2-yl, 3-methyl-3H-imidazo[4,5-b]pyrazin-5-yl, 3-methylphenyl, 3-methyl-pyridin-2-yl, 3-trifluoromethoxyphenyl, 3-trifluoromethylphenyl, 4,5-dimethoxy-pyrimidin-2-yl, 4-amino-5-fluoro-pyrimidin-2-yl, 4-chloro-2,5-dimethoxy-phenyl, 4-chloro-2-fluoro-phenyl, 4-chloro-2-methoxy-5-methyl-phenyl, 4-chloro-pyridin-3-yl, 4-difluoro-2-methyl-phenyl, 4-ethoxy-5-fluoro-pyrimidin-2-yl, 4-ethoxy-pyrimidin-2-yl, 4-ethoxy-pyrimidin-5-yl, 4-ethyl-1H-pyrazol-3-yl, 4-fluoro-2-methoxy-phenyl, 4-fluoro-2-methyl-phenyl, 4-fluorophenyl, 4-methoxy-5-methyl-pyrimidin-2-yl, 4-methoxy-pyridin-3-yl, 4-methoxy-pyrimidin-2-yl, 4-methoxy-pyrimidin-5-yl, 4-methyl-phenyl, 4-methyl-pyridin-2-yl, 4-methyl-pyridin-3-yl, 4-pyrrolidin-1-yl-pyrimidin-2-yl, 5,6-dimethoxy-pyrazin-2-yl, 5-acetyl-thiophen-2-yl, 5-amino-6-ethoxy-pyrazin-2-yl, 5-amino-6-methoxy-3-methyl-pyrazin-2-yl, 5-amino-6-methoxy-pyridin-2-yl, 5-chloro-4-methoxy-pyrimidin-2-yl, 5-chloro-6-methoxy-pyrazin-2-yl, 5-dimethylamino-6-methoxy-pyrazin-2-yl, 5-fluoro-2-methoxyphenyl, 5-fluoro-4-methoxy-pyrimidin-2-yl, 5-fluoro-6-methoxy-pyrazin-2-yl, 5-fluoro-pyridin-2-yl, 5-methoxy-pyridin-3-yl, 5-methoxy-thiophen-2-yl, 5-trifluoromethyl-pyrimidin-2-yl, 6-acetyl-pyridin-2-yl, 6-chloro-pyrazin-2-yl, 6-ethoxy-pyrazin-2-yl, 6-ethoxy-pyridin-2-yl, 6-fluoro-pyridin-2-yl, 6-fluoro-pyridin-3-yl, 6-hydroxy-pyridin-2-yl, 6-methoxy-5-methylamino-pyrazin-2-yl, 6-methoxy-5-methyl-pyrazin-2-yl, 6-methoxy-pyrazin-2-yl, 6-methoxy-pyridin-2-yl, 6-methoxy-pyridin-3-yl, 6-methylamino-pyrazin-2-yl, 6-methyl-pyridin-2-yl, 5-amino-6-(2,2,2-trifluoroethoxy)pyrazin-2-yl, and 6-trifluoromethyl-pyridin-2-yl.

Preferred Hsp90 inhibitor compounds used as the first component of combination according to the invention include:
(R)-2-amino-7-[2-(2-fluoro-pyridin-3-yl)-phenyl]-4-methyl-7,8-dihydro-6H-pyrido[4,3-d]pyrimidin-5-one;
(S)-2-amino-6-benzyl-7-[4-fluoro-2-(2-fluoro-pyridin-3-yl)-phenyl]-4-methyl-7,8-dihydro-6H-pyrido[4,3-d]pyrimidin-5-one;
(R)-2-amino-7-[4-fluoro-2-(2-fluoro-pyridin-3-yl)-phenyl]-4-methyl-7,8-dihydro-6H-pyrido[4,3-d]pyrimidin-5-one;
(R)-2-amino-7-(2-bromo-4-fluoro-phenyl)-6-[(S)-1-(4-methoxy-phenyl)-ethyl]-4-methyl-7,8-dihydro-6H-pyrido[4,3-d]pyrimidin-5-one;
(R)-2-amino-7-[2-(6-methoxy-pyridin-2-yl)-phenyl]-4-methyl-7,8-dihydro-6H-pyrido[4,3-d]pyrimidin-5-one;
(R)-2-amino-7-[4-fluoro-2-(6-methoxy-pyridin-2-yl)-phenyl]-4-methyl-7,8-dihydro-6H-pyrido[4,3-d]pyrimidin-5-one;
2-amino-7-[4-fluoro-2-(6-methoxy-pyridin-2-yl)-phenyl]-4,6-dimethyl-7,8-dihydro-6H-pyrido[4,3-d]pyrimidin-5-one;
2-amino-7-[4-fluoro-2-(2-fluoro-pyridin-3-yl)-phenyl]-4,6-dimethyl-7,8-dihydro-6H-pyrido[4,3-d]pyrimidin-5-one;
2-amino-7-[4-fluoro-2-(6-methoxypyridin-2-yl)phenyl]-4-methyl-7,8-dihydropyrido[4,3-d]pyrimidin-5(6H)-one;
2-amino-7-[2-(6-methoxy-pyrazin-2-yl)-phenyl]-4-methyl-7,8-dihydro-6H-pyrido[4,3-d]pyrimidin-5-one;
(R)-2-amino-7-[4-fluoro-2-(6-methoxy-pyrazin-2-yl)-phenyl]-4-methyl-7,8-dihydro-6H-pyrido[4,3-d]pyrimidin-5-one;
2-amino-7-[4-fluoro-2-(6-methoxy-pyrazin-2-yl)-phenyl]-4,6-dimethyl-7,8-dihydro-6H-pyrido[4,3-d]pyrimidin-5-one;
2-amino-7-[2-(2-methoxy-pyridin-3-yl)-phenyl]-4-methyl-7,8-dihydro-6H-pyrido[4,3-d]pyrimidin-5-one;
2-amino-7-(5,2′-difluoro-biphenyl-2-yl)-4-methyl-7,8-dihydro-6H-pyrido[4,3-d]pyrimidin-5-one;
2-amino-7-(5-fluoro-2′-trifluoromethoxy-biphenyl-2-yl)-4-methyl-7,8-dihydro-6H-pyrido[4,3-d]pyrimidin-5-one;
2-amino-7-[2-(2-chloro-pyridin-3-yl)-4-fluoro-phenyl]-4-methyl-7,8-dihydro-6H-pyrido[4,3-d]pyrimidin-5-one;
2-amino-7-[4-fluoro-2-(6-fluoro-pyridin-3-yl)-phenyl]-4-methyl-7,8-dihydro-6H-pyrido[4,3-d]pyrimidin-5-one;
2-amino-7-(4-fluoro-2-isoquinolin-4-yl-phenyl)-4-methyl-7,8-dihydro-6H-pyrido[4,3-d]pyrimidin-5-one;
2-amino-7-(5,3′-difluoro-biphenyl-2-yl)-4-methyl-7,8-dihydro-6H-pyrido[4,3-d]pyrimidin-5-one;
2-amino-7-[2-(4-chloro-pyridin-3-yl)-4-fluoro-phenyl]-4-methyl-7,8-dihydro-6H-pyrido[4,3-d]pyrimidin-5-one;
2-amino-7-(5,2′-difluoro-3′-methoxy-biphenyl-2-yl)-4-methyl-7,8-dihydro-6H-pyrido[4,3-d]pyrimidin-5-one;
2-amino-7-(5,4′-difluoro-2′-methyl-biphenyl-2-yl)-4-methyl-7,8-dihydro-6H-pyrido[4,3-d]pyrimidin-5-one;
2-amino-7-(5-fluoro-2′-methoxy-biphenyl-2-yl)-4-methyl-7,8-dihydro-6H-pyrido[4,3-d]pyrimidin-5-one;
2-amino-7-(4-fluoro-2-pyrimidin-5-yl-phenyl)-4-methyl-7,8-dihydro-6H-pyrido[4,3-d]pyrimidin-5-one;
2-amino-7-[4-fluoro-2-(2-methoxy-pyridin-3-yl)-phenyl]-4-methyl-7,8-dihydro-6H-pyrido[4,3-d]pyrimidin-5-one;
2-amino-7-(5-fluoro-3′-methoxy-biphenyl-2-yl)-4-methyl-7,8-dihydro-6H-pyrido[4,3-d]pyrimidin-5-one;
(R)-2-amino-6-(3-amino-propyl)-7-[4-fluoro-2-(6-methoxy-pyridin-2-yl)-phenyl]-4-methyl-7,8-dihydro-6H-pyrido[4,3-d]pyrimidin-5-one;
2-amino-7-(4-fluoro-2-pyridin-3-yl-phenyl)-4-methyl-7,8-dihydro-6H-pyrido[4,3-d]pyrimidin-5-one;
2-amino-7-(5,2′-difluoro-4′-methyl-biphenyl-2-yl)-4-methyl-7,8-dihydro-6H-pyrido[4,3-d]pyrimidin-5-one;
2-amino-7-[4-fluoro-2-(1-methyl-1H-pyrazol-4-yl)-phenyl]-4-methyl-7,8-dihydro-6H-pyrido[4,3-d]pyrimidin-5-one;
2-amino-7-[4-fluoro-2-(H-pyrazol-4-yl)-phenyl]-4-methyl-7,8-dihydro-6H-pyrido[4,3-d]pyrimidin-5-one;
2-amino-4-methyl-7-(5,2′,3′-trifluoro-biphenyl-2-yl)-7,8-dihydro-6H-pyrido[4,3-d]pyrimidin-5-one;
2-amino-7-(2-bromo-4-fluoro-phenyl)-4-methyl-6-(2-methyl-2-morpholin-4-yl-propyl)-7,8-dihydro-6H-pyrido[4,3-d]pyrimidin-5-one;
2-amino-7-(3′-dimethylamino-5-fluoro-biphenyl-2-yl)-4-methyl-7,8-dihydro-6H-pyrido[4,3-d]pyrimidin-5-one;
2-amino-7-[2-(2,4-dimethoxy-pyrimidin-5-yl)-4-fluoro-phenyl]-4-methyl-7,8-dihydro-6H-pyrido[4,3-d]pyrimidin-5-one;
2-amino-7-[4-fluoro-2-(5-methoxy-pyridin-3-yl)-phenyl]-4-methyl-7,8-dihydro-6H-pyrido[4,3-d]pyrimidin-5-one;
2-amino-7-(4-fluoro-2-pyrimidin-5-yl-phenyl)-4-methyl-6-(2-methyl-2-morpholin-4-yl-propyl)-7,8-dihydro-6H-pyrido[4,3-d]pyrimidin-5-one;
2-amino-7-[4-fluoro-2-(2-methoxy-pyridin-3-yl)-phenyl]-4-methyl-6-(2-methyl-2-morpholin-4-yl-propyl)-7,8-dihydro-6H-pyrido[4,3-d]pyrimidin-5-one;
2-amino-7-(5-fluoro-3′-methoxy-biphenyl-2-yl)-4-methyl-6-(2-methyl-2-morpholin-4-yl-propyl)-7,8-dihydro-6H-pyrido[4,3-d]pyrimidin-5-one;
(R)-2-amino-7-[4-fluoro-2-(4-methoxy-5-methyl-pyrimidin-2-yl)-phenyl]-4-methyl-7,8-dihydro-6H-pyrido[4,3-d]pyrimidin-5-one;
2-amino-7-(4-fluoro-2-furan-3-yl-phenyl)-4-methyl-7,8-dihydro-6H-pyrido[4,3-d]pyrimidin-5-one, and

stereoisomers, tautomers, and pharmaceutically acceptable salts or prodrugs thereof.

Examples of the foregoing Hsp90 inhibitor compounds of Formula (I) and methods of making the same are disclosed in U.S. Patent Application Publication No. 2007-0123546 A1, published May 31, 2007, which is incorporated herein by reference in its entirety.

The first component can also be an Hsp90 inhibitor of formula (D), or a salt, solvate or hydrate, thereof wherein each R independently represents an optional substituent and R₃ represents a carboxamide group.

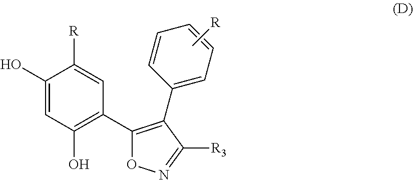

Preferably the present invention relates to the use of compounds consisting of those of formula (E), and regioisomers thereof, and their salts, solvates and hydrates, and prodrugs thereof:

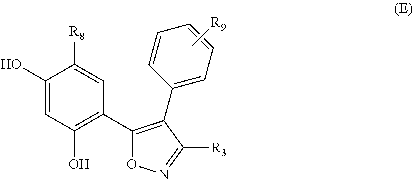

wherein R₃ represents a carboxamide group (such as ethylaminocarbonyl CH₃CH₂NHC(═O)—, or isopropylaminocarbonyl (CH₃)₂CHNHC(═O)—); R₉ represents —CH₂NR¹⁰R¹¹ or —NR¹⁰R¹¹ wherein the substituted amino group —NR¹⁰R¹¹ is a solubilising group, (such as morpholinyl, piperidinyl, piperazinyl, pyrrolidinyl, ethylamino, isopropylamino, diethylamino, cyclohexylamino, cyclopentylamino, methoxyethylamino, piperidin-4-yl, N-acetylpiperazinyl, N-methylpiperazinyl, methylsulfonylamino, thiomorpholinyl, thiomorpholinyl-dioxide, 4-hydroxyethylpiperidinyl, and 4-hydroxypiperidinyl); and R₈ represents an optional substituent, especially a small lipophilic group (such as ethyl, isopropyl, bromo, or chloro).

In such 5-substituted, 2,4-dihydroxy phenyl compounds of the invention, the hydroxyl groups may be protected by groups which are cleaved in the body to release the hydroxyl groups. Known prodrug-type groups of this kind which are cleaved to hydroxyls include alkylcarbonyloxy groups such as methylcarbonyloxy, and alkylaminocarbonyloxy groups such as dialkylamino- or isopropylamino-carbonyloxy.

Specific compounds with which the invention is concerned include particularly the following, and their salts, N-oxides, hydrates and solvates, and prodrugs thereof:
5-(2,4-Dihydroxy-5-isopropyl-phenyl)-4-(4-morpholin-4-ylmethyl-phenyl)-isoxazole-3-carboxylic acid ethylamide
5-(2,4-Dihydroxy-5-isopropyl-phenyl)-4-(4-piperidin-1-ylmethyl-phenyl)-isoxazole-3-carboxylic acid ethylamide
4-(4-Diethylaminomethyl-phenyl)-5-(2,4-dihydroxy-5-isopropyl-phenyl)-isoxazole-3-carboxylic acid ethylamide
5-(2,4-Dihydroxy-5-isopropyl-phenyl)-4-[4-(4-methyl-piperazin-1-ylmethyl)-phenyl]-isoxazole-3-carboxylic acid ethylamide
5-(2,4-Dihydroxy-5-isopropyl-phenyl)-4-(4-ethylaminomethyl-phenyl)-isoxazole-3-carboxylic acid ethylamide
5-(2,4-Dihydroxy-5-isopropyl-phenyl)-4-[4-(isopropylamino-methyl)-phenyl]-isoxazole-3-carboxylic acid ethylamide
4-(4-Cyclohexylaminomethyl-phenyl)-5-(2,4-dihydroxy-5-isopropyl-phenyl)-isoxazole-3-carboxylic acid ethylamide
4-[4-(tert-Butylamino-methyl)-phenyl]-5-(2,4-dihydroxy-5-isopropyl-phenyl)-isoxazole-3-carboxylic acid ethylamide
5-(2,4-Dihydroxy-5-isopropyl-phenyl)-4-{4-[(2-methoxy-ethylamino)-methyl]-phenyl}-isoxazole-3-carboxylic acid ethylamide
5-(2,4-Dihydroxy-5-isopropyl-phenyl)-4-(4-morpholin-4-ylmethyl-phenyl)-isoxazole-3-carboxylic acid isopropylamide
5-(2,4-Dihydroxy-5-isopropyl-phenyl)-4-[4-(4-methyl-piperazin-1-ylmethyl)-phenyl]-isoxazole-3-carboxylic acid isopropylamide
5-(5-tert-Butyl-2,4-dihydroxy-phenyl)-4-[4-(4-methyl-piperazin-1-ylmethyl)-phenyl]-isoxazole-3-carboxylic acid ethylamide
5-(5-tert-Butyl-2,4-dihydroxy-phenyl)-4-(4-piperidin-1-ylmethyl-phenyl)-isoxazole-3-carboxylic acid ethylamide
5-(2,4-Dihydroxy-5-isobutyl-phenyl)-4-(4-morpholin-4-ylmethyl-phenyl)-isoxazole-3-carboxylic acid ethylamide
5-(2,4-Dihydroxy-5-isobutyl-phenyl)-4-(4-piperidin-1-ylmethyl-phenyl)-isoxazole-3-carboxylic acid ethylamide
5-(5-tert-Butyl-2,4-dihydroxy-phenyl)-4-(4-morpholin-4-ylmethyl-phenyl)-isoxazole-3-carboxylic acid ethylamide
5-(5-tert-Butyl-2,4-dihydroxy-phenyl)-4-(4-diethylaminomethyl-phenyl)-isoxazole-3-carboxylic acid ethylamide
3-(5-Chloro-2,4-dihydroxy-phenyl)-4-(4-morpholin-4-ylmethyl-phenyl)-isoxazole-5-carboxylic acid ethylamide
4-(4-Diethylaminomethyl-phenyl)-5-(4,6-dihydroxy-2′-methyl-biphenyl-3-yl)-isoxazole-3-carboxylic acid ethylamide
4-(4-Diethylaminomethyl-phenyl)-5-(4′-fluoro-4,6-dihydroxy-biphenyl-3-yl) isoxazole-3-carboxylic acid ethylamide
4-(4-Diethylaminomethyl-phenyl)-5-(4,6-dihydroxy-biphenyl-3-yl)-isoxazole-3-carboxylic acid ethylamide
5-(2′-Fluoro-4,6-dihydroxy-biphenyl-3-yl)-4-(4-pyrrolidin-1-ylmethyl-phenyl)-isoxazole-3-carboxylic acid ethylamide
5-(4,6-Dihydroxy-biphenyl-3-yl)-4-(4-morpholin-4-ylmethyl-phenyl)-isoxazole-3-carboxylic acid ethylamide
5-(2,4-Dihydroxy-5-phenethyl-phenyl)-4-(4-morpholin-4-ylmethyl-phenyl)-isoxazole-3-carboxylic acid ethylamide
5-(5-Chloro-2,4-dihydroxy-phenyl)-4-(4-piperidin-1-ylmethyl-phenyl)-isoxazole-3-carboxylic acid isopropylamide
4-(4-Diethylaminomethyl-phenyl)-5-(5-ethyl-2,4-dihydroxy-phenyl)-isoxazole-3-carboxylic acid ethylamide
5-(5-Ethyl-2,4-dihydroxy-phenyl)-4-[4-(4-methyl-piperazin-1-ylmethyl)-phenyl]-isoxazole-3-carboxylic acid ethylamide
5-(5-Ethyl-2,4-dihydroxy-phenyl)-4-(4-morpholin-4-ylmethyl-phenyl)-isoxazole-3-carboxylic acid ethylamide
5-(5-Chloro-2,4-dihydroxy-phenyl)-4-(4-diethylaminomethyl-phenyl)-isoxazole-3-carboxylic acid ethylamide
5-(5-Chloro-2,4-dihydroxy-phenyl)-4-[4-(4-methyl-piperazin-1-ylmethyl)-phenyl]-isoxazole-3-carboxylic acid ethylamide
5-(5-Chloro-2,4-dihydroxy-phenyl)-4-(4-morpholin-4-ylmethyl-phenyl)-isoxazole-3-carboxylic acid ethylamide

Compounds within the scope of formula (D) or formula (E) and the process for their manufacture are disclosed in WO 04/072051 published on Aug. 26, 2004 which is hereby incorporated into the present application by reference. A preferred compound is 5-(2,4-Dihydroxy-5-isopropyl-phenyl)-4-(4-morpholin-4-ylmethyl-phenyl)-isoxazole-3-carboxylic acid ethylamide.

The present invention provides a pharmaceutical combination comprising

a) a compound of formula (E)

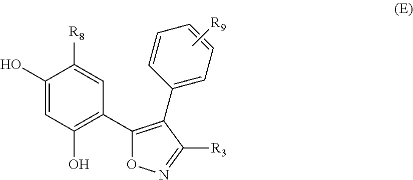

wherein R₃ is selected from ethylaminocarbonyl CH₃CH₂NHC(═O)— or isopropylaminocarbonyl (CH₃)₂CHNHC(═O)—),

R₈ is selected from ethyl, isopropyl, bromo, or chloro; and

R₉ is selected from morpholinyl, piperidinyl, piperazinyl, pyrrolidinyl, ethylamino, isopropylamino, diethylamino, cyclohexylamino, cyclopentylamino, methoxyethylamino, piperidin-4-yl, N-acetylpiperazinyl, N-methylpiperazinyl, methylsulfonylamino, thiomorpholinyl, thiomorpholinyl-dioxide, 4-hydroxyethylpiperidinyl or 4-hydroxypiperidinyl; and

b) at least one HER2 (ErbB2) inhibitor

The compound of formula (E) may be a Hsp90 inhibitor.

The compound of formula (E) may be 5-(2,4-Dihydroxy-5-isopropyl-phenyl)-4-(4-morpholin-4-ylmethyl-phenyl)-isoxazole-3-carboxylic acid ethylamide.

In another aspect the present invention provides the use of a compound of formula (E) or 5-(2,4-Dihydroxy-5-isopropyl-phenyl)-4-(4-morpholin-4-ylmethyl-phenyl)-isoxazole-3-carboxylic acid ethylamide and at least one HER2 (ErbB2) inhibitor for the manufacture of a medicament for the treatment or prevention of a proliferative disease.

In a further aspect the present invention provides a compound of formula (E) or 5-(2,4-Dihydroxy-5-isopropyl-phenyl)-4-(4-morpholin-4-ylmethyl-phenyl)-isoxazole-3-carboxylic acid ethylamide and at least one HER2 (ErbB2) inhibitor for use in treating or preventing a proliferative disease.

In another aspect the present invention provides a method of treating or preventing a proliferative disease by administering a compound of formula (E) or 5-(2,4-Dihydroxy-5-isopropyl-phenyl)-4-(4-morpholin-4-ylmethyl-phenyl)-isoxazole-3-carboxylic acid ethylamide and at least one HER2 (ErbB2) inhibitor.

The present invention further relates to combinations comprising compounds targeting, decreasing or inhibiting the activity of the epidermal growth factor (EGFR) family of receptor tyrosine kinases (ErbB1 (EGFR1), ErbB2 (EGFR2, HER2), ErbB3 (EGFR3), ErbB4 (EGFR4) as homo- or heterodimers), such as compounds which target, decrease or inhibit the activity of the epidermal growth factor receptor family are especially compounds, proteins or antibodies which inhibit members of the EGF receptor tyrosine kinase family, e.g., EGF receptor, ErbB1, ErbB2, ErbB3 and ErbB4 or bind to EGF or EGF-related ligands, and are in particular those compounds, proteins or monoclonal antibodies generically and specifically disclosed in U.S. Pat. No. 5,677,171, which is hereby incorporated into the present application by reference. Comprised are likewise the corresponding stereoisomers as well as the corresponding crystal modifications, e.g. solvates and polymorphs, which are disclosed therein. More preferred is trastuzumab also marketed under the tradename Herceptin™. Trastuzumab, a monoclonal antibody, works by interfering with one of the ways in which breast cancer cells grow and divide. Some breast cancer cells overexpress a protein known as HER2 (ErbB2) on their cell surface. Trastuzumab blocks the positive growth signal HER2 overexpression supplies to the tumor by attaching itself to the HER2 (ErbB2) protein and acting as a HER2 (ErbB2) inhibitor. This inhibits tumor cell division and growth. Trastuzumab also works by attracting the body's own immune cells to help destroy the cancer cells. Trastuzumab is approved for the treatment of breast cancer whose tumors overexpress the HER2 (ErbB2) protein. Another example of a HER2 (ErbB2) inhibitor is lapatinib. An example of a HER1 (ErbB1) inhibitor is erlotinib.

In each case where citations of patent applications are given above, the subject matter relating to the compounds is hereby incorporated into the present application by reference. Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the invention can be prepared and administered as described in the cited documents, respectively. Also within the scope of this invention is the combination of more than two separate active ingredients as set forth above, i.e., a pharmaceutical combination within the scope of this invention could include three active ingredients or more.

In another embodiment the present invention provides the use of the first pharmaceutical component of the present invention in combination with a HER2 inhibitor for the manufacture of a medicament for the treatment or prevention of a proliferative disease.

In a further embodiment the present invention provides the first pharmaceutical component of the present invention in combination with a HER2 inhibitor for use in treating or preventing a proliferative disease.

Suitable clinical studies may be, for example, open label, dose escalation studies in patients with proliferative diseases. Such studies prove in particular the synergism of the active ingredients of the combination of the invention. The beneficial effects on proliferative diseases may be determined directly through the results of these studies which are known as such to a person skilled in the art. Such studies may be, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) may be administered in a fixed dose and the dose of agent (b) may be escalated. Each patient may receive doses of the agent (a) either daily or intermittent. The efficacy of the treatment may be determined in such studies, e.g., after 12, 18 or 24 weeks by evaluation of symptom scores every 6 weeks.

The administration of a pharmaceutical combination of the invention may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically active ingredients used in the combination of the invention.

A further benefit may be that lower doses of the active ingredients of the combination of the invention may be used, for example, that the dosages need not only often be smaller but may also be applied less frequently, which may diminish the incidence or severity of side-effects. This is in accordance with the desires and requirements of the patients to be treated.

It is one objective of this invention to provide a pharmaceutical composition comprising a quantity of a first component and a second component as previously described, which may be jointly therapeutically effective at targeting or preventing proliferative diseases. These first and second components may be provided for administration in a fixed combination, i.e. in a single galenical composition, which may be prepared in a manner known per se, suitable for enteral, such as oral or rectal, and parenteral administration to mammals (warm-blooded animals), including humans in combination with one or more pharmaceutically acceptable carriers or diluents, especially suitable for enteral or parenteral application.

Alternatively, the first component and the second component may be provided as separate pharmaceutical compositions in a kit.

The pharmaceutical compositions for separate administration of the first component and the second component may be prepared in a manner known per se and are those suitable for enteral, such as oral or rectal, and parenteral administration to mammals (warm-blooded animals), including humans. Each such composition for separate administration comprises a therapeutically effective amount of at least one pharmacologically active component in combination with one or more pharmaceutically acceptable carriers or diluents.

Suitable pharmaceutical compositions may contain, for example, from about 0.1% to about 99.9%, preferably from about 1% to about 60%, of the active ingredient(s). Pharmaceutical preparations for the combination therapy for enteral or parenteral administration are, for example, those in unit dosage forms, such as sugar-coated tablets, tablets, capsules or suppositories, or ampoules. If not indicated otherwise, these are prepared in a manner known per se, for example by means of conventional mixing, granulating, sugar-coating, dissolving or lyophilizing processes.

It will be appreciated that the unit content of a pharmaceutical component contained in an individual dose of each dosage form need not in itself constitute an effective amount since the necessary effective amount may be reached by administration of a plurality of dosage units.

In a method of treating proliferative disease, the first component and the second component may be administered together, sequentially or separately. The first and second components may be delivered in one combined unit dosage form or in multiple separate unit dosage forms.

In particular, a therapeutically effective amount of each of the pharmaceutical components of the invention may be administered simultaneously or sequentially and in any order, and the components may be administered separately or as a fixed combination. For example, the method of preventing or treating proliferative diseases according to the invention may comprise (i) administration of the first component in free or pharmaceutically acceptable salt form and (ii) administration of the second component in free or pharmaceutically acceptable salt form, simultaneously or sequentially in any order, in jointly therapeutically effective amounts, preferably in synergistically effective amounts, e.g. in daily or intermittently dosages corresponding to the amounts described herein. The individual combination components of the combination of the invention may be administered separately at different times during the course of therapy or concurrently in divided or single combination forms. Furthermore, the term administering also encompasses the use of a pro-drug of a combination component that convert in vivo to the combination partner as such. The instant invention is therefore to be understood as embracing all such regimens of simultaneous or alternating treatment and the term “administering” is to be interpreted accordingly.

The effective dosage of each of the components employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including the route of administration and the renal and hepatic function of the patient. A clinician or physician of ordinary skill can readily determine and prescribe the effective amount of the single active ingredients required to alleviate, counter or arrest the progress of the condition. Optimal precision in achieving concentration of the active ingredients within the range that yields efficacy without toxicity requires a regimen based on the kinetics of the active ingredients' availability to target sites.

The amount of active ingredient that may be combined with carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

For purposes of the present invention, a therapeutically effective dose will generally be a total daily dose administered to a host in single or divided doses may be in amounts, for example, of from 0.001 to 1000 mg/kg body weight daily and more preferred from 1.0 to 30 mg/kg body weight daily. Dosage unit compositions may contain such amounts of submultiples thereof to make up the daily dose.

The compounds according to Formula (I), the HER2 inhibitors and the pharmaceutical compositions comprising these active ingredients, may be administered orally, parenterally, sublingually, by aerosolization or inhalation spray, rectally, or topically in dosage unit formulations containing conventional nontoxic pharmaceutically acceptable carriers, adjuvants, and vehicles as desired. Topical administration may also involve the use of transdermal administration such as transdermal patches or ionophoresis devices. The term parenteral as used herein includes subcutaneous injections, intravenous, intramuscular, intrasternal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols, which are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds according to Formula (I), HER2 inhibitors and pharmaceutical compositions described herein can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott (ed.), “Methods in Cell Biology,” Volume XIV, Academic Press, New York, 1976, p. 33 et seq.

The terms “proliferative disease” and “proliferative disorder” include but are not restricted to cancer, e.g. solid tumors, e.g. breast cancer.

### EXAMPLES

### Example 1

Antitumor effect of compound I 5-(2,4-Dihydroxy-5-isopropyl-phenyl)-4-(4-morpholin-4-ylmethyl-phenyl)-isoxazole-3-carboxylic acid ethylamide when used in combination with trastuzumab in a human breast carcinoma xenograft model in nude mice

The estrogen receptor positive cell line BT-474 (ATCC, number HTB-20) which is a human breast carcinoma cell line is used. The cells are grown in DMEM high glucose (4.5 g/l) supplemented with 10% FCS, 200 mM L-glutamine and 1% sodium pyruvate.

In preparation for cell inoculation, each mouse is subcutaneously implanted on the upper dorsal side with a 17β-Estradiol pellet (25 microgram/day; 90 day release) using a trocar needle. BT-474 cells (5×10̂6) are injected in 200 microliter Matrigel:HBSS (1:1 vol) (BD Matrigel™ Basement Membrane Matrix). The injection site is subcutaneously in the right flank. Treatment with compound I is initiated when the average tumor volume reached approximately 100 mm³. Tumor growth is monitored at regular intervals. The xenograft tumor sizes is measured manually with calipers and the tumor volume is estimated using the formula: (W×L²×π/6), where width (W) and length (L) are the two largest diameters. Results are presented as mean±SEM. Tumor data are analyzed by ANOVA with post hoc Dunnet's test for comparison of treatment versus control group.

As a measure of efficacy the % T/C value is calculated at the end of the experiment according to:

(Δtumor volume_{treated}/Δtumor volume_{control})*100

Where Δtumor volumes represent the mean tumor volume on the evaluation day minus the mean tumor volume at the start of the experiment.

Tumor regression is presented as the relative absolute change in tumor volume from the start to the end of the experiment (−(Tumor volume_{evaluation day}−Tumor volume_{start day})/Tumor volume_{start day}*100).

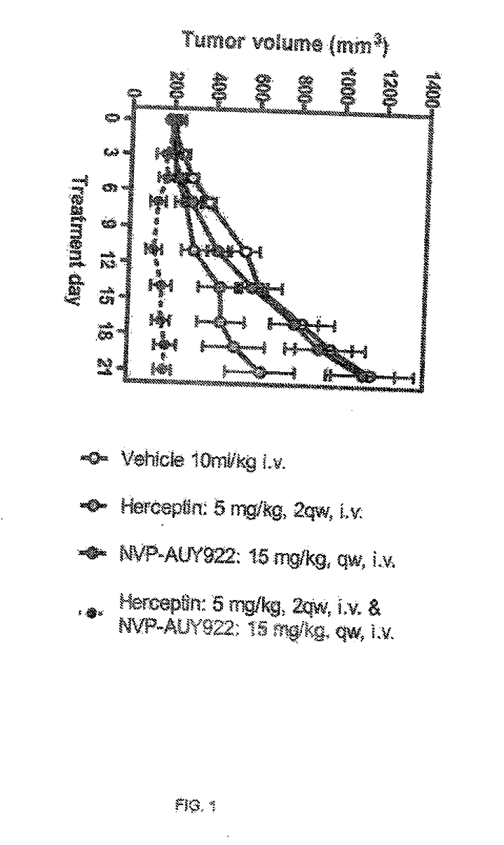

The antitumor effect of compound I and trastazumab is evaluated in the BT-474 xenograft model (FIG. 1). The treatment period is 21 days and each treatment group consists of 8 tumor bearing animals. The tumor sizes in the treatment groups are compared to those of the vehicle treated groups and the effect is expressed as % T/C or regression when applicable. Trastuzumab is administered intravenously (i.v.) twice per week at a dose level of 5 mg/kg. Compound I is administered i.v. once per week at a dose of 15 mg/kg.

Table I shows that 5 mg/kg trastuzumab which is administered i.v. twice per week results in 57% reduction of tumor burden, Compound I which is administered once per week (qw) at a dose of 15 mg/kg gives a 4% reduction in tumor burden (no statistically significant difference from vehicle treated animals). The combination of the two agents results in a strongly enhanced antitumor effect compared with either one alone as 22% tumor regression was observed. These data provide strong pre-clinical support for combining trastuzumab with compound I against ERBB2 positive breast cancer.

TABLE 1

| | | | | ΔTumor |
|---|---|---|---|---|
| | | T/C | Regresssion | volume |
| Compound | Dose, schedule, route | (%) | (%) | (mm³) |
| Vehicle | 10 ml/kg, qw, i.v. | 100 | — | 970 ± 208 |
| control | | | | |
| Trastuzumab | 5 mg/kg, 2qw, i.v. | 43 | — | 415 ± 155 |
| Compound I | 15 mg/kg, qw, i.v. | 96 | — | 929 ± 152 |
| Compound I + | 15 mg/kg, qw, i.v. | — | 22 | −48 ± 31* |
| Trastuzumab | 5 mg/kg, 2qw, i.v. | | | |

*P < 0.05; one-way ANOVA post hoc Dunnet's test.

What is claimed is:

1. A pharmaceutical combination comprising an HSP90 inhibitor and a HER2 inhibitor, wherein the HSP90 inhibitor is
(A) a compound of formula (E) 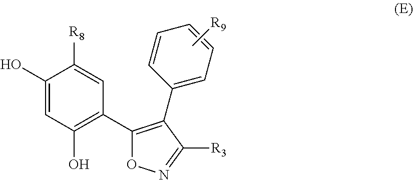
wherein
R₃ is selected from ethylaminocarbonyl CH₃CH₂NHC(═O)— or isopropylaminocarbonyl (CH₃)₂CHNHC(═O)—),
R₈ is selected from ethyl, isopropyl, bromo, or chloro; and
R₉ is —CH₂NR¹⁰R¹¹ or —NR¹⁰R¹¹ wherein the substituted amino group —NR¹⁰R¹¹ is a morpholinyl, piperidinyl, piperazinyl, pyrrolidinyl, ethylamino, isopropylamino, diethylamino, cyclohexylamino, cyclopentylamino, methoxyethylamino, piperidin-4-yl, N-acetylpiperazinyl, N-methylpiperazinyl, methylsulfonylamino, thiomorpholinyl, thiomorpholinyl-dioxide, 4-hydroxyethylpiperidinyl or 4-hydroxypiperidinyl, or pharmaceutically acceptable salt or prodrug thereof;
or (B) compound according to Formula (I) 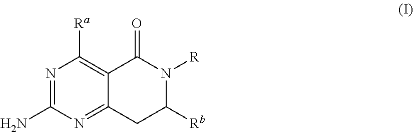
wherein R^{a} is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C1-C6 alkyl,
(8) amino or substituted amino,
(9) substituted or unsubstituted aryl,
(10) substituted or unsubstituted heteroaryl, and
(11) substituted or unsubstituted heterocyclyl;
R is selected from the group consisting of
(1) hydrogen,
(2) substituted or unsubstituted C1-C6 alkyl,
(3) substituted or unsubstituted C2-C6 alkenyl,
(4) substituted or unsubstituted C2-C6 alkynyl,
(5) substituted or unsubstituted C3-C7 cycloalkyl,
(6) substituted or unsubstituted C5-C7 cycloalkenyl,
(7) substituted or unsubstituted aryl,
(8) substituted or unsubstituted heteroaryl, and
(9) substituted or unsubstituted heterocyclyl;
R^{b} is selected from the group consisting of
(1) substituted or unsubstituted C3-C7 cycloalkyl,
(2) substituted or unsubstituted C5-C7 cycloalkenyl,
(3) substituted or unsubstituted aryl,
(4) substituted or unsubstituted heteroaryl, and
(5) substituted or unsubstituted heterocyclyl; and
with the proviso that when R^{a} is amino, then R^{b} is not phenyl, 4-alkyl-phenyl, 4-alkoxy-phenyl, or 4-halo-phenyl, or a pharmaceutically acceptable salt or prodrug thereof.

2. The pharmaceutical combination according to claim 1, wherein the HER2 inhibitor is trastuzumab

3. A pharmaceutical combination of claim 1, wherein the HSP90 inhibitor is a compound according to formula (III) 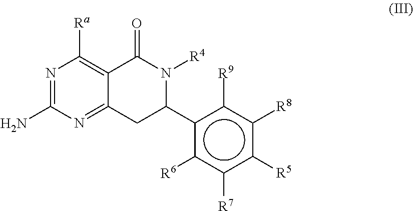
wherein R^{a} is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C1-C6 alkyl,
(8) amino or substituted amino,
(9) substituted or unsubstituted aryl,
(10) substituted or unsubstituted heteroaryl, and
(1) substituted or unsubstituted heterocyclyl;
R⁴ is hydrogen or substituted or unsubstituted C1-C6 alkyl;
R⁵ is hydrogen, alkyl, alkoxy, or halo;
each of R⁶, R⁷, R⁸, and R⁹ are independently selected from the group consisting of hydrogen, alkyl, alkoxy, halo, substituted or unsubstituted aryl, and substituted or unsubstituted heteroaryl;
with the proviso that when R^{a} is amino and R⁶, R⁷, R⁸, and R⁹ are hydrogen, then R⁵ is not hydrogen, alkyl, alkoxy, or halo;
or a pharmaceutically acceptable salt or prodrug thereof.

4. The pharmaceutical combination according to claim 3, wherein the HER2 inhibitor is trastuzumab.

5. A pharmaceutical combination comprising, a HSP90 inhibitor and a HER2 inhibitor, wherein the HSP90 inhibitor is:
(R)-2-amino-7-[2-(2-fluoro-pyridin-3-yl)-phenyl]-4-methyl-7,8-dihydro-6H-pyrido[4,3-d]pyrimidin-5-one,
(S)-2-amino-6-benzyl-7-[4-fluoro-2-(2-fluoro-pyridin-3-yl)-phenyl]-4-methyl-7,8-dihydro-6H-pyrido[4,3-d]pyrimidin-5-one,
(R)-2-amino-7-[4-fluoro-2-(2-fluoro-pyridin-3-yl)-phenyl]-4-methyl-7,8-dihydro-6H-pyrido[4,3-d]pyrimidin-5-one,
(R)-2-amino-7-(2-bromo-4-fluoro-phenyl)-6-[(S)-1-(4-methoxy-phenyl)-ethyl]-4-methyl-7,8-dihydro-6H-pyrido[4,3-d]pyrimidin-5-one,
(R)-2-amino-7-[2-(6-methoxy-pyridin-2-yl)-phenyl]-4-methyl-7,8-dihydro-6H-pyrido[4,3-d]pyrimidin-5-one,
(R)-2-amino-7-[4-fluoro-2-(6-methoxy-pyridin-2-yl)-phenyl]-4-methyl-7,8-dihydro-6H-pyrido[4,3-d]pyrimidin-5-one,
2-amino-7-[4-fluoro-2-(6-methoxy-pyridin-2-yl)-phenyl]-4,6-dimethyl-7,8-dihydro-6H-pyrido[4,3-d]pyrimidin-5-one,
2-amino-7-[4-fluoro-2-(2-fluoro-pyridin-3-yl)-phenyl]-4,6-dimethyl-7,8-dihydro-6H-pyrido[4,3-d]pyrimidin-5-one,
2-amino-7-[4-fluoro-2-(6-methoxypyridin-2-yl)phenyl]-4-methyl-7,8-dihydropyrido[4,3-d]pyrimidin-5(6H)-one,
2-amino-7-[2-(6-methoxy-pyrazin-2-yl)-phenyl]-4-methyl-7,8-dihydro-6H-pyrido[4,3-d]pyrimidin-5-one,
(R)-2-amino-7-[4-fluoro-2-(6-methoxy-pyrazin-2-yl)-phenyl]-4-methyl-7,8-dihydro-6H-pyrido[4,3-d]pyrimidin-5-one,
2-amino-7-[4-fluoro-2-(6-methoxy-pyrazin-2-yl)-phenyl]-4,6-dimethyl-7,8-dihydro-6H-pyrido[4,3-d]pyrimidin-5-one,
2-amino-7-[2-(2-methoxy-pyridin-3-yl)-phenyl]-4-methyl-7,8-dihydro-6H-pyrido[4,3-d]pyrimidin-5-one,
2-amino-7-(5,2′-difluoro-biphenyl-2-yl)-4-methyl-7,8-dihydro-6H-pyrido[4,3-d]pyrimidin-5-one,
2-amino-7-(5-fluoro-2′-trifluoromethoxy-biphenyl-2-yl)-4-methyl-7,8-dihydro-6H-pyrido[4,3-d]pyrimidin-5-one,
2-amino-7-[2-(2-chloro-pyridin-3-yl)-4-fluoro-phenyl]-4-methyl-7,8-dihydro-6H-pyrido[4,3-d]pyrimidin-5-one,
2-amino-7-[4-fluoro-2-(6-fluoro-pyridin-3-yl)-phenyl]-4-methyl-7,8-dihydro-6H-pyrido[4,3-d]pyrimidin-5-one,
2-amino-7-(4-fluoro-2-isoquinolin-4-yl-phenyl)-4-methyl-7,8-dihydro-6H-pyrido[4,3-d]pyrimidin-5-one,
2-amino-7-(5,3′-difluoro-biphenyl-2-yl)-4-methyl-7,8-dihydro-6H-pyrido[4,3-d]pyrimidin-5-one,
2-amino-7-[2-(4-chloro-pyridin-3-yl)-4-fluoro-phenyl]-4-methyl-7,8-dihydro-6H-pyrido[4,3-d]pyrimidin-5-one,
2-amino-7-(5,2′-difluoro-3′-methoxy-biphenyl-2-yl)-4-methyl-7,8-dihydro-6H-pyrido[4,3-d]pyrimidin-5-one,
2-amino-7-(5,4′-difluoro-2′-methyl-biphenyl-2-yl)-4-methyl-7,8-dihydro-6H-pyrido[4,3-d]pyrimidin-5-one,
2-amino-7-(5-fluoro-2′-methoxy-biphenyl-2-yl)-4-methyl-7,8-dihydro-6H-pyrido[4,3-d]pyrimidin-5-one,
2-amino-7-(4-fluoro-2-pyrimidin-5-yl-phenyl)-4-methyl-7,8-dihydro-6H-pyrido[4,3-d]pyrimidin-5-one,
2-amino-7-[4-fluoro-2-(2-methoxy-pyridin-3-yl)-phenyl]-4-methyl-7,8-dihydro-6H-pyrido[4,3-d]pyrimidin-5-one,
2-amino-7-(5-fluoro-3′-methoxy-biphenyl-2-yl)-4-methyl-7,8-dihydro-6H-pyrido[4,3-d]pyrimidin-5-one,
(R)-2-amino-6-(3-amino-propyl)-7-[4-fluoro-2-(6-methoxy-pyridin-2-yl)-phenyl]-4-methyl-7,8-dihydro-6H-pyrido[4,3-d]pyrimidin-5-one,
2-amino-7-(4-fluoro-2-pyridin-3-yl-phenyl)-4-methyl-7,8-dihydro-6H-pyrido[4,3-d]pyrimidin-5-one,
2-amino-7-(5,2′-difluoro-4′-methyl-biphenyl-2-yl)-4-methyl-7,8-dihydro-6H-pyrido[4,3-d]pyrimidin-5-one,
2-amino-7-[4-fluoro-2-(1-methyl-1H-pyrazol-4-yl)-phenyl]-4-methyl-7,8-dihydro-6H-pyrido[4,3-d]pyrimidin-5-one,
2-amino-7-[4-fluoro-2-(1H-pyrazol-4-yl)-phenyl]-4-methyl-7,8-dihydro-6H-pyrido[4,3-d]pyrimidin-5-one,
2-amino-4-methyl-7-(5,2′,3′-trifluoro-biphenyl-2-yl)-7,8-dihydro-6H-pyrido[4,3-d]pyrimidin-5-one,
2-amino-7-(2-bromo-4-fluoro-phenyl)-4-methyl-6-(2-methyl-2-morpholin-4-yl-propyl)-7,8-dihydro-6H-pyrido[4,3-d]pyrimidin-5-one,
2-amino-7-(3′-dimethylamino-5-fluoro-biphenyl-2-yl)-4-methyl-7,8-dihydro-6H-pyrido[4,3-d]pyrimidin-5-one,
2-amino-7-[2-(2,4-dimethoxy-pyrimidin-5-yl)-4-fluoro-phenyl]-4-methyl-7,8-dihydro-6H-pyrido[4,3-d]pyrimidin-5-one,
2-amino-7-[4-fluoro-2-(5-methoxy-pyridin-3-yl)-phenyl]-4-methyl-7,8-dihydro-6H-pyrido[4,3-d]pyrimidin-5-one,
2-amino-7-(4-fluoro-2-pyrimidin-5-yl-phenyl)-4-methyl-6-(2-methyl-2-morpholin-4-yl-propyl)-7,8-dihydro-6H-pyrido[4,3-d]pyrimidin-5-one,
2-amino-7-[4-fluoro-2-(2-methoxy-pyridin-3-yl)-phenyl]-4-methyl-6-(2-methyl-2-morpholin-4-yl-propyl)-7,8-dihydro-6H-pyrido[4,3-d]pyrimidin-5-one,
2-amino-7-(5-fluoro-3′-methoxy-biphenyl-2-yl)-4-methyl-6-(2-methyl-2-morpholin-4-yl-propyl)-7,8-dihydro-6H-pyrido[4,3-d]pyrimidin-5-one,
(R)-2-amino-7-[4-fluoro-2-(4-methoxy-5-methyl-pyrimidin-2-yl)-phenyl]-4-methyl-7,8-dihydro-6H-pyrido[4,3-d]pyrimidin-5-one,
2-amino-7-(4-fluoro-2-furan-3-yl-phenyl)-4-methyl-7,8-dihydro-6H-pyrido[4,3-d]pyrimidin-5-one,
5-(2,4-Dihydroxy-5-isopropyl-phenyl)-4-(4-morpholin-4-ylmethyl-phenyl)-isoxazole-3-carboxylic acid ethylamide,
or a pharmaceutically salt thereof.

6. A method of treating a proliferative disease comprising administering to a patient in need thereof an effective amount of a first pharmaceutical agent and a second pharmaceutical agent, wherein the first pharmaceutical agent is (A) a compound of formula (E) 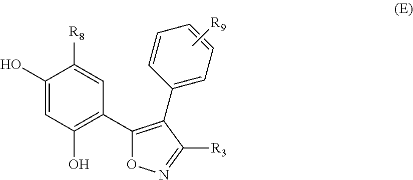
wherein
R₃ is selected from ethylaminocarbonyl CH₃CH₂NHC(═O)— or isopropylaminocarbonyl (CH₃)₂CHNHC(═O)—),
R₈ is selected from ethyl, isopropyl, bromo, or chloro; and
R₉ is —CH₂NR¹⁰R¹¹ or —NR¹⁰R¹¹ wherein the substituted amino group —NR¹⁰R¹¹ is a morpholinyl, piperidinyl, piperazinyl, pyrrolidinyl, ethylamino, isopropylamino, diethylamino, cyclohexylamino, cyclopentylamino, methoxyethylamino, piperidin-4-yl, N-acetylpiperazinyl, N-methylpiperazinyl, methylsulfonylamino, thiomorpholinyl, thiomorpholinyl-dioxide, 4-hydroxyethylpiperidinyl or 4-hydroxypiperidinyl or a pharmaceutically acceptable salt or prodrug thereof; or
(B) a compound according to formula (III) 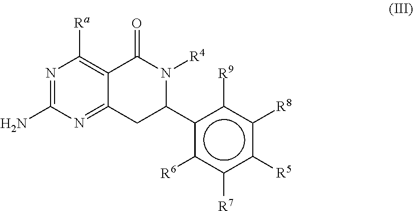
or a pharmaceutically acceptable salt or prodrug thereof, wherein
wherein R^{a} is selected from the group consisting of
(1) hydrogen,
(2) halogen,
(3) hydroxyl,
(4) C₁-C₆ alkoxy,
(5) thiol,
(6) C₁-C₆ alkylthiol,
(7) substituted or unsubstituted C1-C6 alkyl,
(8) amino or substituted amino,
(9) substituted or unsubstituted aryl,
(10) substituted or unsubstituted heteroaryl, and
(11) substituted or unsubstituted heterocyclyl;
R⁴ is hydrogen or substituted or unsubstituted C1-C6 alkyl;
R⁵ is hydrogen, alkyl, alkoxy, or halo;
each of R⁶, R⁷, R⁸, and R⁹ are independently selected from the group consisting of hydrogen, alkyl, alkoxy, halo, substituted or unsubstituted aryl, and substituted or unsubstituted heteroaryl;
with the proviso that when R^{a} is amino and R⁶, R⁷, R⁸, and R⁹ are hydrogen, then R⁵ is not hydrogen, alkyl, alkoxy, or halo or a pharmaceutically acceptable salt or prodrug thereof and the second pharmaceutical component is a HER2 inhibitor.

7. The method of claim 6, wherein the HER2 inhibitor is trastuzumab.

8. The method of claim 6, wherein the first pharmaceutical agent is
(R)-2-amino-7-[2-(2-fluoro-pyridin-3-yl)-phenyl]-4-methyl-7,8-dihydro-6H-pyrido[4,3-d]pyrimidin-5-one,
(S)-2-amino-6-benzyl-7-[4-fluoro-2-(2-fluoro-pyridin-3-yl)-phenyl]-4-methyl-7,8-dihydro-6H-pyrido[4,3-d]pyrimidin-5-one,
(R)-2-amino-7-[4-fluoro-2-(2-fluoro-pyridin-3-yl)-phenyl]-4-methyl-7,8-dihydro-6H-pyrido[4,3-d]pyrimidin-5-one,
(R)-2-amino-7-(2-bromo-4-fluoro-phenyl)-6-[(S)-1-(4-methoxy-phenyl)-ethyl]-4-methyl-7,8-dihydro-6H-pyrido[4,3-d]pyrimidin-5-one,
(R)-2-amino-7-[2-(6-methoxy-pyridin-2-yl)-phenyl]-4-methyl-7,8-dihydro-6H-pyrido[4,3-d]pyrimidin-5-one,
(R)-2-amino-7-[4-fluoro-2-(6-methoxy-pyridin-2-yl)-phenyl]-4-methyl-7,8-dihydro-6H-pyrido[4,3-d]pyrimidin-5-one,
2-amino-7-[4-fluoro-2-(6-methoxy-pyridin-2-yl)-phenyl]-4,6-dimethyl-7,8-dihydro-6H-pyrido[4,3-d]pyrimidin-5-one,
2-amino-7-[4-fluoro-2-(2-fluoro-pyridin-3-yl)-phenyl]-4,6-dimethyl-7,8-dihydro-6H-pyrido[4,3-d]pyrimidin-5-one,
2-amino-7-[4-fluoro-2-(6-methoxypyridin-2-yl)phenyl]-4-methyl-7,8-dihydropyrido[4,3-d]pyrimidin-5(6H)-one,
2-amino-7-[2-(6-methoxy-pyrazin-2-yl)-phenyl]-4-methyl-7,8-dihydro-6H-pyrido[4,3-d]pyrimidin-5-one,
(R)-2-amino-7-[4-fluoro-2-(6-methoxy-pyrazin-2-yl)-phenyl]-4-methyl-7,8-dihydro-6H-pyrido[4,3-d]pyrimidin-5-one,
2-amino-7-[4-fluoro-2-(6-methoxy-pyrazin-2-yl)-phenyl]-4,6-dimethyl-7,8-dihydro-6H-pyrido[4,3-d]pyrimidin-5-one,
2-amino-7-[2-(2-methoxy-pyridin-3-yl)-phenyl]-4-methyl-7,8-dihydro-6H-pyrido[4,3-d]pyrimidin-5-one,
2-amino-7-(5,2′-difluoro-biphenyl-2-yl)-4-methyl-7,8-dihydro-6H-pyrido[4,3-d]pyrimidin-5-one,
2-amino-7-(5-fluoro-2′-trifluoromethoxy-biphenyl-2-yl)-4-methyl-7,8-dihydro-6H-pyrido[4,3-d]pyrimidin-5-one,
2-amino-7-[2-(2-chloro-pyridin-3-yl)-4-fluoro-phenyl]-4-methyl-7,8-dihydro-6H-pyrido[4,3-d]pyrimidin-5-one,
2-amino-7-[4-fluoro-2-(6-fluoro-pyridin-3-yl)-phenyl]-4-methyl-7,8-dihydro-6H-pyrido[4,3-d]pyrimidin-5-one,
2-amino-7-(4-fluoro-2-isoquinolin-4-yl-phenyl)-4-methyl-7,8-dihydro-6H-pyrido[4,3-d]pyrimidin-5-one,
2-amino-7-(5,3′-difluoro-biphenyl-2-yl)-4-methyl-7,8-dihydro-6H-pyrido[4,3-d]pyrimidin-5-one,
2-amino-7-[2-(4-chloro-pyridin-3-yl)-4-fluoro-phenyl]-4-methyl-7,8-dihydro-6H-pyrido[4,3-d]pyrimidin-5-one,
2-amino-7-(5,2′-difluoro-3′-methoxy-biphenyl-2-yl)-4-methyl-7,8-dihydro-6H-pyrido[4,3-d]pyrimidin-5-one,
2-amino-7-(5,4′-difluoro-2′-methyl-biphenyl-2-yl)-4-methyl-7,8-dihydro-6H-pyrido[4,3-d]pyrimidin-5-one,
2-amino-7-(5-fluoro-2′-methoxy-biphenyl-2-yl)-4-methyl-7,8-dihydro-6H-pyrido[4,3-d]pyrimidin-5-one,
2-amino-7-(4-fluoro-2-pyrimidin-5-yl-phenyl)-4-methyl-7,8-dihydro-6H-pyrido[4,3-d]pyrimidin-5-one,
2-amino-7-[4-fluoro-2-(2-methoxy-pyridin-3-yl)-phenyl]-4-methyl-7,8-dihydro-6H-pyrido[4,3-d]pyrimidin-5-one,
2-amino-7-(5-fluoro-3′-methoxy-biphenyl-2-yl)-4-methyl-7,8-dihydro-6H-pyrido[4,3-d]pyrimidin-5-one,
(R)-2-amino-6-(3-amino-propyl)-7-[4-fluoro-2-(6-methoxy-pyridin-2-yl)-phenyl]-4-methyl-7,8-dihydro-6H-pyrido[4,3-d]pyrimidin-5-one,
2-amino-7-(4-fluoro-2-pyridin-3-yl-phenyl)-4-methyl-7,8-dihydro-6H-pyrido[4,3-d]pyrimidin-5-one,
2-amino-7-(5,2′-difluoro-4′-methyl-biphenyl-2-yl)-4-methyl-7,8-dihydro-6H-pyrido[4,3-d]pyrimidin-5-one,
2-amino-7-[4-fluoro-2-(1-methyl-1H-pyrazol-4-yl)-phenyl]-4-methyl-7,8-dihydro-6H-pyrido[4,3-d]pyrimidin-5-one,
2-amino-7-[4-fluoro-2-(1H-pyrazol-4-yl)-phenyl]-4-methyl-7,8-dihydro-6H-pyrido[4,3-d]pyrimidin-5-one,
2-amino-4-methyl-7-(5,2′,3′-trifluoro-biphenyl-2-yl)-7,8-dihydro-6H-pyrido[4,3-d]pyrimidin-5-one,
2-amino-7-(2-bromo-4-fluoro-phenyl)-4-methyl-6-(2-methyl-2-morpholin-4-yl-propyl)-7,8-dihydro-6H-pyrido[4,3-d]pyrimidin-5-one,
2-amino-7-(3′-dimethylamino-5-fluoro-biphenyl-2-yl)-4-methyl-7,8-dihydro-6H-pyrido[4,3-d]pyrimidin-5-one,
2-amino-7-[2-(2,4-dimethoxy-pyrimidin-5-yl)-4-fluoro-phenyl]-4-methyl-7,8-dihydro-6H-pyrido[4,3-d]pyrimidin-5-one,
2-amino-7-[4-fluoro-2-(5-methoxy-pyridin-3-yl)-phenyl]-4-methyl-7,8-dihydro-6H-pyrido[4,3-d]pyrimidin-5-one,
2-amino-7-(4-fluoro-2-pyrimidin-5-yl-phenyl)-4-methyl-6-(2-methyl-2-morpholin-4-yl-propyl)-7,8-dihydro-6H-pyrido[4,3-d]pyrimidin-5-one,
2-amino-7-[4-fluoro-2-(2-methoxy-pyridin-3-yl)-phenyl]-4-methyl-6-(2-methyl-2-morpholin-4-yl-propyl)-7,8-dihydro-6H-pyrido[4,3-d]pyrimidin-5-one,
2-amino-7-(5-fluoro-3′-methoxy-biphenyl-2-yl)-4-methyl-6-(2-methyl-2-morpholin-4-yl-propyl)-7,8-dihydro-6H-pyrido[4,3-d]pyrimidin-5-one,
(R)-2-amino-7-[4-fluoro-2-(4-methoxy-5-methyl-pyrimidin-2-yl)-phenyl]-4-methyl-7,8-dihydro-6H-pyrido[4,3-d]pyrimidin-5-one,
2-amino-7-(4-fluoro-2-furan-3-yl-phenyl)-4-methyl-7,8-dihydro-6H-pyrido[4,3-d]pyrimidin-5-one,
5-(2,4-Dihydroxy-5-isopropyl-phenyl)-4-(4-morpholin-4-ylmethyl-phenyl)-isoxazole-3-carboxylic acid ethylamide,
or a pharmaceutically acceptable salt thereof.

9. The method according to claim 6, wherein treating said proliferative disorder comprises treatment of solid tumors.

10. The method according to claim 9, wherein said proliferative disorder is breast cancer.

11. A method of treating a proliferative disease comprising administering to a patient in need thereof an effective amount of an Hsp 90 inhibitor and an HER2 inhibitor for therapeutic treatment of the proliferative disease.

12. The combination of claim 1, wherein the combination is a kit.

13. The combination of claim 1, wherein the combination is a pharmaceutical composition.

14. The method according to claim 6, wherein the compound according to Formula (III) and the HER2 inhibitor are administered simultaneously, concurrently, separately, or sequentially for treating a proliferative disorder.
